(19) United States
(12) Patent Application Publication
(10) Pub. No.: US 2012/0134981 A1
(43) Pub. Date: May 31, 2012

(54) GENES LINKING SEVERAL COMPLICATIONS OF TYPE-2 DIABETES (T2D)

(75) Inventors: Pavel HAMET, Ville Mont-Royal (CA); Johanne Tremblay, Ville Mont-Royal (CA); Ondrej Seda, Prague (CZ); Stephen MaCmahon, Sydney (AU); John Chalmers, Sydney (AU)

(73) Assignee: PROGNOMIX, INC., Montreal (CA)

(21) Appl. No.: 13/237,707

(22) Filed: Sep. 20, 2011

Related U.S. Application Data
(60) Provisional application No. 61/384,602, filed on Sep. 20, 2010.

(51) Int. Cl. A61K 39/395; A61P 9/12; A61K 31/713; A61P 9/10; C40B 20/00; A61K 31/7088

(52) U.S. Cl. 424/130.1; 506/2; 514/44 A

(57) ABSTRACT

The invention provides means and methods to predict, in subjects affected by type II diabetes (T2D), the probability of developing complications which include, but are not limited to, micro/macrovascular disorder, hypertension, neuropathy, atrial fibrillation, nephropathy and other major adverse cardiovascular events (MACE) that are associated with the disease, by detecting one or more genetic features. The genetic features that are useful in prediction include, but are not limited to, genes, single nucleotide polymorphisms (SNPs) and other genomic markers. The invention further involves characterizing individuals based on the probability of developing complications related to T2D, such as, micro/macrovascular disorder, hypertension, neuropathy, atrial fibrillation, nephropathy or MACE, based on the identification of one or more aforementioned genetic features. Also described are combinations and kits for carrying out the above-described methods.

This application claims the benefit of U.S. provisional Application No. 61/384,602, filed on Sep. 20, 2010, the disclosure of which is incorporated herein by reference in its entirety.

The invention provides means and methods to predict, in subjects affected by type II diabetes (T2D), the probability of developing complications related to the disease.

The invention further provides methods for identifying subjects that have one or more genetic features linked to T2D-related complications. These complications include, but are not limited to, micro/macrovascular disorder, hypertension, neuropathy, atrial fibrillation, nephropathy and other major adverse cardiovascular events (MACE). The genetic features that are useful in characterizing subjects include, but are not limited to, genes, single nucleotide polymorphisms (SNPs) and other genomic markers. The invention further involves characterizing individuals based on the probability of developing complications related to T2D, such as, micro/macrovascular disorder, hypertension, neuropathy, atrial fibrillation, nephropathy, or MACE, based on the identification of one or more aforementioned genetic features. Also described are combinations and kits for carrying out the above-described methods.

Diabetes mellitus is a heterogeneous group of metabolic diseases which is characterized by elevated blood glucose levels and increased morbidity. The endocrine cells of the pancreas which synthesize insulin and other hormones are involved in the pathogenesis of diabetes. Both genetic and environmental factors contribute to its development. The most common form is T2D, which is characterized by defects in both insulin secretion and insulin action, e.g., insulin resistance. In contrast, type I diabetes results from autoimmune destruction of the insulin-producing beta cells of the pancreas. Monogenic forms of diabetes account for less than 5% of the cases and are usually caused by mutations in genes associated with maturity-onset diabetes of the young (MODY), insulin gene and insulin receptor gene.

The prevalence of diabetes for all age-groups worldwide was estimated to be 2.8% in 2000 and 4.4% in 2030 (Wild S, Roglic G, Green A, Sicree R, and King H. Global prevalence of diabetes. Diabetes Care, 27(5):1047-53, 2004). In the past two decades alone there has been a dramatic increase in the diagnosis of T2D. T2D is a chronic metabolic disease that has a significant impact on the health, quality of life, and life expectancy of patients, as well as on the health care system. T2D and its complications are multifactorial traits and known to have a strong genetic component with contributing environmental determinants. Micro- and macrovascular disorder can affect many organs and can lead to the development of severe complications including nephropathy, neuropathy, retinopathy and major adverse cardiovascular events (MACE) in about half of the patients suffering from T2D. In the present study, we used the ADVANCE cohort that is a randomized trial of blood pressure lowering and intensive glucose control on vascular outcomes in high-risk individuals with T2D mellitus to identify the genetic determinants of T2D complications (See Patel et al. “Effect of a fixed combination of perindopril and indapamide on micro/macrovascular and microvascular outcomes in patients with type 2 diabetes mellitus (The ADVANCE trial): a randomised controlled trial.” Lancet, 370(9590):829-40, 2007; Patel et al. “Intensive blood glucose control and vascular outcomes in patients with type 2 diabetes.” N Engl J Med 2008 Jun. 12; 358(24):2560-72; Zoungas et al. “Combined effects of routine blood pressure lowering and intensive glucose control on macrovascular and microvascular outcomes in patients with type 2 diabetes: New results from the ADVANCE trial.” Diabetes Care 2009 November; 32(11):2068-74).

A variety of environmental factors such as obesity or sedentary lifestyle and diabetogenic genes are thought to contribute to the development of T2D. Abnormal glucose homeostasis occurs when either insulin sensitivity or insulin secretion or both are altered. An early finding in this development is insulin resistance, defined as impaired insulin-mediated glucose clearance in insulin-sensitive tissues (skeletal muscle, liver and adipose tissue). Elevation of glucose levels triggers beta-cells to produce and secrete more insulin, which compensates for the disturbance in glucose homeostasis. The duration of hyperglycemia-hyperinsulinemia state depends on insulin secretory capacity, mass and apoptosis rate of beta-cells. Furthermore, beta-cells can lose their insulin secretion capacity because of glucose toxicity or other reasons. When cells fail to compensate for insulin resistance, blood glucose concentration increases. Thus, over time subclinical hyperglycemia tends to progress to impaired glucose tolerance and further to T2D.

The causes of T2D are multi-factorial and include both genetic and environmental elements that affect beta cell function and insulin sensitivity of peripheral tissues (muscle, liver, adipose tissue, and pancreas). Although there is considerable debate as to the relative contributions of beta-cell dysfunction and reduced insulin sensitivity to the pathogenesis of diabetes, it is generally agreed that both of these factors play important roles. Both impaired insulin secretion and insulin action cause the development of T2D. Insulin resistance is an early feature in the pathophysiology of T2D.

No major single gene explaining the development of T2D has been identified. There were several studies attempting to predict T2D based on limited number of SNPs (up to 18) [Lyssenko, V, P Almgren, D Anevski et al.: Genetic prediction of future type 2 diabetes. PLoS Med 2, e345 (2005); Meigs, J B, P Shrader, LM Sullivan et al.: Genotype score in addition to common risk factors for prediction of type 2 diabetes. N Engl J Med 359, 2208-19 (2008); Cauchi, S, D Meyre, E Durand et al.: Post genome-wide association studies of novel genes associated with type 2 diabetes show gene-gene interaction and high predictive value. PLoS ONE 3, e2031 (2008). Miyake, K, W Yang, K Hara et al.: Construction of a prediction model for type 2 diabetes mellitus in the Japanese population based on 11 genes with strong evidence of the association. J Hum Genet 54, 236-41 (2009); Lin, X, K Song, N Lim et al.: Risk prediction of prevalent diabetes in a Swiss population using a weighted genetic score—the CoLaus Study. Diabetologia 52, 600-8 (2009)] but the achieved predictive power was generally limited. Indeed, the mathematical models reflecting the presumable allele frequencies and risk effects estimate that for successful prediction, hundreds of SNPs will be necessary [(Kraft, P, D J Hunter: Genetic Risk Prediction—Are We There Yet? N Engl J Med 360, 1701-1703 (2009)].

Complications Associated with Diabetes

Over 90% of people diagnosed with diabetes have T2D, which carries a number of potential complications. These complications currently add very significantly to the cost of treating diabetes, because there is no reliable way to determine which patients are likely to develop such difficulties. Majority of the people affected by T2D die from complications resulting from the disease.

Such complications include, but are not limited to:

Cardiovascular Disease

Cardiovascular disease is the overwhelming cause of diabetes-related deaths. With the risk for stroke or myocardial infarction elevated by 2 to 4 times in persons with diabetes, a 65% majority of deaths among people with diabetes occurs from heart disease or stroke, considered as major micro/macrovascular complications.

Hypertension

Diabetes and high blood pressure are closely related disorders. They occur together so frequently that they are officially considered to be co-morbidities. Diabetes makes high blood pressure more difficult to treat, and high blood pressure makes diabetes even more dangerous. Studies have shown that 5% of patients have high blood pressure within 10 years, 33% have high blood pressure within 20 years and 70% have high blood pressure by 40 years of diabetes since its diagnosis. Moreover, almost 75% of T2D patients with kidney problems (a common complication) had high blood pressure. In T2D patients that had no incidence of kidney problems, the rate of high blood pressure is about 40%. Overall, when averaged across diabetes type and age range, about 35% of all people with diabetes have high blood pressure.

Atrial Fibrillation

Results from a recent study suggest that individuals with diabetes are at an increased risk of developing atrial fibrillation (Dublin et al. Diabetes mellitus, glycemic control, and risk of atrial fibrillation. J Gen Intern Med 2010). In this study, the researchers used data from Group Health, a large, integrated healthcare system in the US. Included in this population-based case-control analysis were 1410 people with newly recognized atrial fibrillation and 2203 controls without atrial fibrillation. Of those with atrial fibrillation, 39% had transitory atrial fibrillation, defined as a single episode lasting seven days or less and not recurring in the next six months. Among individuals with atrial fibrillation, 18% had diabetes treated by their physicians with medications, while 14% of controls had pharmacologically treated diabetes. This translated into a significant 30% increased risk of atrial fibrillation among those with treated diabetes, even after adjustment for body-mass index, among other variables. The study also showed that this risk was higher among patients with a longer duration of treated diabetes and poorer glycemic control.

Major Adverse Cardiovascular Events (MACE)

MACE is defined as occurrence of one or more of cardiovascular death, non fatal myocardial infarction or non-fatal stroke. It is also tightly linked to T2D.

Diabetic Nephropathy

End-stage renal disease (ESRD) occurs when the kidneys cease to function, which ultimately leads to the need for a transplant or regular dialysis, both extremely costly procedures. Diabetes is responsible for 43% of the cases of ESRD as a consequence of microvascular damage of the kidney.

Diabetic Retinopathy

Diabetes is also the leading cause of blindness in people aged 20-74. Diabetic retinopathy is considered as one type of microvascular complication and is responsible for over 24,000 cases of blindness in the United States.

Diabetic Neuropathy

It is estimated that over 70% of people with diabetes may also suffer from nervous system damage, causing impaired sensation or pain in the feet or hands, slowed digestion of food in the stomach, carpal tunnel syndrome, and other nerve problems. In the severe cases of diabetic neuropathy, usually combined with peripheral vessel macro and microvascular disease, patients may have to undergo lower-extremity amputations.

Albuminuria

Albuminuria is a pathological condition wherein albumin is present in the urine. It is a type of proteinuria, well demonstrated precursor of renal failure but also of myocardial infarction and stroke, well demonstrated precursor of renal failure but also of myocardial infarction and stroke.

Dental disease, complications of pregnancy, coma, and acute susceptibility to opportunistic infectious diseases are also costly diabetes-related diseases.

Drugs designed to prevent or stabilize complications are extremely costly. These drugs also have various side effects associated with using them. Because of the debilitating effects of diabetes-related complications, and despite the side effects of the medications themselves, healthcare professionals must prescribe costly medications to diabetes patients to protect them against developing these complications without having any efficient and reliable mean to predict those patients who will develop these complications and the efficiency of these treatments, as well as which patients might be more likely to benefit from the treatments.

There is a need for assays capable of identifying among T2D patients those who are at risk of developing complications and those who would benefit from the various treatments available. The present invention provides with means to develop such assays and to utilize them in a clinical and medical environment.

Polymorphisms

DNA polymorphisms provide an efficient way to study the association of genes and diseases by analysis of linkage and linkage disequilibrium. With the sequencing of the human genome a myriad of hitherto unknown genetic polymorphisms among people have been detected. Most common among these are the single nucleotide polymorphisms, also called SNPs, of which there are known several millions. Other examples are short tandem repeat polymorphisms (STR), variable number of tandem repeat polymorphisms (VNTR), insertions, deletions and block modifications. Tandem repeats (STR or VNTR) often have multiple different alleles (variants) in population, whereas the other groups of polymorphisms usually have just two alleles. Some of these genetic polymorphisms play a direct role in the biology of the individuals, including their risk of developing disease, but the virtue of the majority is that they can serve as markers for the surrounding DNA. The relationship of an allele of one sequence polymorphism with particular alleles of other sequence polymorphisms in the surrounding is due to phenomenon called genetic linkage. Linkage arises because large parts of chromosomes are passed unchanged from parents to offspring, so that minor regions of a chromosome tend to flow unchanged from one generation to the next and also to be similar in different branches of the same family. Linkage is gradually eroded by recombination occurring in the germline cells, but typically operates over multiple generations and distances of a number of million bases in the DNA.

Linkage disequilibrium deals with whole populations and has its origin in the (distant) forefather in whose DNA a new sequence polymorphism arose. The immediate surroundings in the DNA of the forefather will tend to stay with the new allele and propagate together to the offspring for many generations. Recombination and changes in the composition of the population will again erode the association, but the new allele and the alleles of any other polymorphism nearby will often be partly associated among unrelated humans even today. A crude estimate suggests that alleles of sequence polymorphisms with distances less than 10000 bases in the DNA will have tended to stay together since modern man arose. Linkage disequilibrium in limited populations, for instance Europeans, often extends over longer distances, e.g. over more than 1,000,000 bases. This can be the result of newer mutations, but can also be a consequence of one or more “bottlenecks” with small effective population sizes and considerable inbreeding in the history of the current population. Two obvious possibilities for “bottlenecks” in Europeans are the exodus from Africa and the repopulation of Europe after the last ice age. A number of polymorphisms have been associated with induction of exocrine pancreatic dysfunction and/or diabetes. Some of the identified polymorphisms have been suggested in patent literature as useful in diagnosis of diabetes (see for example WO9321343 related to polymorphisms in GCK gene, and WO0023591 related to polymorphism in ZSIG49 gene).

Several genomic regions or individual genetic variants are known to be linked or associated to the phenotypes closely related to diabetic complications.

Linkage Studies

In a recent study (Diabetes. 2008 January; 57(1):235-43), a genome-wide scan for estimated glomerular filtration rate was performed in multi-ethnic diabetic populations (the Family Investigation of Nephropathy and Diabetes (FIND)) using 404 STR markers. For all ethnicities combined, strong evidence for linkage was observed on chromosomes 1q43, 7q36.1, 8q13.3 and 18q23.3. Mexican-American families, who comprised the major ethnic subpopulation in FIND, contributed to linkage on chromosomes 1q43, 2p13.3, 7q36.1, 8q13.3, and 18q23.3, whereas African-American and American-Indian families displayed linkage peaks on chromosomes 11p15.1 and 15q22.3, respectively. Also in FIND study (Diabetes. 2007 June; 56(6):1577-85), the strongest evidence of linkage to the diabetic nephropathy trait was on chromosomes 7q21.3, 10p15.3, 14q23.1, and 18q22.3. In ACR (883 diabetic sibling pairs), the strongest linkage signals were on chromosomes 2q14.1, 7q21.1, and 15q26.3. These results confirm regions of linkage to diabetic nephropathy on chromosomes 7q, 10p, and 18q from prior reports. In Mexican Americans, Puppala al. (Diabetes. 2007 November; 56 (11):2818-28) found a linkage of glomerular filtration rate to a region on chromosome 2q near marker D2S427 (corrected LOD score 3.3), influenced by genotype by diabetes interaction.

Association Studies

A number of genes and genetic polymorphisms were tested for their association to diabetic nephropathy, either because of their reported relevance in metabolic and signaling pathways connected to pathophysiology of diabetic complications (functional candidates) or combination of the former with their genomic position under peak of ascertained linkage (positional candidates), or as a result of genome-wide association studies. Genes for which an association was found with diabetic nephropathy include 5,10-methylenetetrahydrofolate reductase (MTHFR), natriuretic peptide precursor A (NPPA), solute carrier family 2 member 1 (facilitated glucose transporter SLC2A1), lamin A/C (LMNA), retinoid X receptor gamma (RXRG), interleukin 1 receptor antagonist (IL1 RN), ghrelin/obestatin preprohormone (GHRL), peroxisome proliferator-activated receptor gamma (PPARG), chemokine receptor 5 (CCR5), angiotensin II receptor type 1 (AGTR1), solute carrier family 2 member 2 (facilitated glucose transporter SLC2A2), adiponectin (ADIPOQ), fatty acid binding protein 2 (FABP2), glutamine-fructose-6-phosphate transaminase 2 (GFPT2), advanced glycosylation end product-specific receptor (AGER), lymphotoxin alpha (LTA), vascular endothelial growth factor A (VEGF), ectonucleotide pyrophosphatase/phosphodiesterase 1 (ENPP1), SMT3 suppressor of mif two 3 homolog 4 (small ubiquitin-like modifier 4 protein SUM04), estrogen receptor 1 (ESR1), superoxide dismutase 2 (SOD2), neuropeptide Y (NPY), engulfment and cell motility 1 (ELMO1), insulin-like growth factor binding protein 1 (IGFBP1), epidermal growth factor receptor (EGFR), paraoxonase 1 (PON1), aldo-keto reductase family 1, member B1 (AKR1B1), caldesmon 1 (CALD1), nitric oxide synthase 3 (NOS3), lipoprotein lipase (LPL), Pvt1 oncogene homolog MYC activator (PVT1), insulin (INS), xylosyltransferase I (XYLT), protein kinase C beta 1 (PRKCB1), solute carrier family 12 (SLC12A3), haptoglobin (HP), chemokine ligand 2 (CCL2), angiotensin I converting enzyme (ACE), meprin A, beta (MEP1B), carnosine dipeptidase 1 (CNDP1), intercellular adhesion molecule 1 (ICAM1), transforming growth factor beta 1 (TGFB1), apolipoprotein E (APOE) and superoxide dismutase 1 (SOD1), CD48 molecule (CD48), solute carrier family 35, member F3 (SLC35F3), endothelial PAS domain protein 1 (EPAS1), low density lipoprotein-related protein 1B (deleted in tumours) (LRP1B), dynein, axonemal, heavy chain 7 (DNAH7), ADAM metallopeptidase domain 23 (ADAM23), leprecan-likel (LEPREL1), human immunodeficiency virus type I enhancer binding protein 2 (HIVEP2), thrombospondin, type I, domain containing 7A (THSD7A), 5-adenosylhomocysteine hydrolase-like 2 (AHCYL2), discs, large homolog 2 (Drosophila) (DLG2), FYVE, RhoGEF and PH domain containing 4 (FGD4), rabphilin 3A homolog (mouse) (RPH3A), citrate lyase beta like (CLYBL), protein kinase C, eta (PRKCH), epidermal growth factor receptor pathway substrate 15-like 1 (EPS15L1), cystatin 9 (testatin) (CST9), pericentrin (PCNT2), matrix metallopeptidase 9 (gelatinase B, 92 kDa gelatinase, 92 kDa type IV collagenase) (MMP9), apolipoprotein C-I (APOC1), cysteinyl-tRNA synthetase (CARS), chimerin (chimaerin) 2 (CHN2), neurocalcin delta (NCALD), ectonucleotide pyrophosphatase/phosphodiesterase 1 (ENPP1), major histocompatibility complex, class II, DR beta 1 (HLA-DRB1), interleukin 8 (IL8), pleckstrin homology domain containing, family H (with MyTH4 domain) member 2 (PLEKHH2), angiotensinogen (serpin peptidase inhibitor, Glade A, member 8) (AGT) and others, reviewed in Maeda, S: Genetics of diabetic nephropathy. Ther Adv Cardiovasc Dis 2, 363-71 (2008).

Genes and markers in type-2 diabetes (T2D)-related complications are described in US patent application pub. No. 2010-0099091, US patent application pub. No. 2010-0136540 and PCT application pub. No. WO/2009/150550 (all to Hamet et al.), the disclosures of which are incorporated by reference in their entirety.

Proteomic Studies

Few proteomic studies have been so far conducted with the aim of identifying biomarkers of predictive value for diabetic nephropathy. Otu et al. (Diabetes Care (2007) 30:638-543) (WO 2007/056587) performed a nested case-control study on Pima Indians that allowed for identification of a 12-peak proteomic signature that was validated on a small number of individuals. However, the study did not permit the identification of the proteins from which these peaks belong and replication in other populations is needed prior to concluding the broad applicability of these biomarkers.

There have been dozens of genetic variants and genome regions associated or linked to myocardial infarction, both in T2D patients as well as in non-diabetics. These have been comprehensively reviewed recently (Yamada, Y, S Ichihara, T Nishida: Molecular genetics of myocardial infarction. Genomic Med 2, 7-22 (2008)) and specifically for genome-wide association studies in A Catalog of Published Genome-Wide Association Studies by The National Human Genome Research Institute (available on the world wide web at genome.gov/26525384) Johnson, A D, C J O'Donnell: An open access database of genome-wide association results. BMC Med Genet 10, 6 (2009).

### SUMMARY OF THE INVENTION

The present invention relates to previously unknown associations between various polymorphisms, genes and loci and T2D-related complications. These associated polymorphisms, genes, and loci are associated to at least two phenotypes of T2D and provide basis for novel methods and kits for risk assessment, diagnosis and prognosis of T2D-related complication in a patient, as well as related inventions. In addition, the identification of these polymorphisms, genes and loci provide the basis for methods and kits for novel therapies to prevent, treat and/or reduce risk of developing these complications.

A “biomarker” in the context of the present invention refers to a genetic feature such as, for example, single nucleotide polymorphism (SNP) or a short tandem repeat (STR). Other types of biomarkers include, but are not limited to, transcriptional products (such as, for example, mRNA or cDNA sequences thereof) or translational products (such as, for example, proteins or polypeptides) of genes comprising such SNPs. Representative examples of such SNPs are disclosed in Tables 1-4. Particularly preferred are SNPs having the REFSNPID (RS) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099, rs6698441, or a combination thereof.

Tables 1-4: Lists of Common Genes Associated to 3 or More Complications of T2D.

PDE4D: Phosphodiesterase 4D, cAMP-Specific

### Chromosome: 5

Location: 5q12

TABLE 1
SNPs of PDE4D associated to 9 phenotypes with odd ratios and their positions

| | | | | Sex-averaged | |
|---|---|---|---|---|---|
| | | | Minor | Marshfield | |
| Phenotype | SNP ID | O.R. | Allele | position (cM) | Flanking sequence |
| Have | rs10051847 | 0.7304 | T | 68.682203 | ATGCATTCTCTATTCA[C/T]TCCTGTTCCTTTTTTT |
| albuminuria | | | | | |
| Macrovascular | rs10051847 | 0.7356 | T | 68.682203 | ATGCATTCTCTATTCA[C/T]TCCTGTTCCTTTTTTT |
| disorder | rs11951359 | 0.7447 | G | 68.683876 | CTGTAAACTTCTATAC[G/T]ACTTTGTACCACCCAT |
| | rs2968005 | 1.3068 | G | 68.702057 | TGGTGAAACCTGGTTG[G/T]ACTGAACCTTTAGGAG |
| Micro_Macro- | rs10051847 | 0.6547 | T | 68.682203 | ATGCATTCTCTATTCA[C/T]TCCTGTTCCTTTTTTT |
| vascular | rs11951359 | 0.6547 | G | 68.683876 | CTGTAAACTTCTATAC[G/T]ACTTTGTACCACCCAT |
| disorder | rs12657171 | 0.4714 | A | 68.690560 | TGCTGAGTGAACACAA[A/G]CAGTAACCTATGTTCT |
| | rs1077183 | 0.4497 | C | 68.702526 | CATAGATCTTATAAAA[C/G]TTTAACTGAGCATTAG |
| | rs10514870 | 0.5448 | G | 68.716308 | TCTATCTTTTGTCAGC[A/G]AGTTTCCTCAGAGTTC |
| | rs10066573 | 0.4198 | T | 68.718363 | TTAACCTCGGACTGCC[A/T]ACTTTCAGATTTTACA |
| | rs10068543 | 0.4350 | C | 68.718694 | TATATGAACATTTGTA[C/T]CATTCAACTGTCTAAC |
| | rs6450502 | 0.4215 | T | 68.720227 | GTTGGATCTCTATTAA[A/T]CCTCTCTAAAATGTCA |
| | rs6864156 | 0.4340 | C | 68.723430 | GGAAGCTCTGTTTGCA[C/T]AGGTAAGATTTGTCAA |
| | rs33927508 | 0.5475 | G | 68.747053 | ATCAACACACATTTAA[A/G]GCATATCAGTTTTACC |
| | rs16889615 | 0.7525 | A | 68.835443 | AATGAATCTTGCCTTA[A/G]GCTACATAAATTACAA |
| | rs10471476 | 0.8046 | G | 69.238089 | TTCAGTGTGCCCAGAC[G/T]AGAACTCATCTTCCCT |
| MI_Angina | rs10051847 | 0.6383 | T | 68.682203 | ATGCATTCTCTATTCA[C/T]TCCTGTTCCTTTTTTT |
| | rs11951359 | 0.6383 | G | 68.683876 | CTGTAAACTTCTATAC[G/T]ACTTTGTACCACCCAT |
| | rs12657171 | 0.4852 | A | 68.690560 | TGCTGAGTGAACACAA[A/G]CAGTAACCTATGTTCT |
| | rs1077183 | 0.4750 | C | 68.702526 | CATAGATCTTATAAAA[C/G]TTTAACTGAGCATTAG |
| MACE | rs10051847 | 0.6160 | T | 68.682203 | ATGCATTCTCTATTCA[C/T]TCCTGTTCCTTTTTTT |
| | rs11951359 | 0.6160 | G | 68.683876 | CTGTAAACTTCTATAC[G/T]ACTTTGTACCACCCAT |
| | rs12657171 | 0.4840 | A | 68.690560 | TGCTGAGTGAACACAA[A/G]CAGTAACCTATGTTCT |
| | rs1077183 | 0.4752 | C | 68.702526 | CATAGATCTTATAAAA[C/G]TTTAACTGAGCATTAG |
| MACE + | rs829258 | 0.7359 | C | 68.679974 | TTTACATATATGTAAG[C/G]ACATTTGGTTTATCTT |
| Albuminuria | rs10051847 | 0.6363 | T | 68.682203 | ATGCATTCTCTATTCA[C/T]TCCTGTTCCTTTTTTT |
| | rs11951359 | 0.6363 | G | 68.683876 | CTGTAAACTTCTATAC[G/T]ACTTTGTACCACCCAT |
| | rs10461656 | 0.7267 | A | 68.684196 | TGGCTTTGAGTTTGTA[A/G]AGGTATGCATGGCCTC |
| | rs12657171 | 0.5901 | A | 68.690560 | TGCTGAGTGAACACAA[A/G]CAGTAACCTATGTTCT |
| | rs1077183 | 0.4348 | C | 68.702526 | CATAGATCTTATAAAA[C/G]TTTAACTGAGCATTAG |
| | rs10514870 | 0.5274 | G | 68.716308 | TCTATCTTTTGTCAGC[A/G]AGTTTCCTCAGAGTTC |
| | rs10066573 | 0.4468 | T | 68.718363 | TTAACCTCGGACTGCC[A/T]ACTTTCAGATTTTACA |
| | rs10068543 | 0.4621 | C | 68.718694 | TATATGAACATTTGTA[C/T]CATTCAACTGTCTAAC |
| | rs6450502 | 0.4508 | T | 68.720227 | GTTGGATCTCTATTAA[A/T]CCTCTCTAAAATGTCA |
| | rs6864156 | 0.4612 | C | 68.723430 | GGAAGCTCTGTTTGCA[C/T]AGGTAAGATTTGTCAA |
| Hypertension | rs829258 | 0.7059 | C | 68.679974 | TTTACATATATGTAAG[C/G]ACATTTGGTTTATCTT |
| | rs10051847 | 0.6104 | T | 68.682203 | ATGCATTCTCTATTCA[C/T]TCCTGTTCCTTTTTTT |
| | rs11951359 | 0.6104 | G | 68.683876 | CTGTAAACTTCTATAC[G/T]ACTTTGTACCACCCAT |
| | rs10461656 | 0.7060 | A | 68.684196 | TGGCTTTGAGTTTGTA[A/G]AGGTATGCATGGCCTC |
| Atrial | rs16889508 | 1.6639 | A | 68.807957 | CTGCTACATTACCTAC[A/G]AGTTTATCCCAATCCA |
| Fibrillation | rs17528550 | 1.7904 | A | 68.809484 | GAGTGAAGATGGTAAA[A/G]CCAGAAAGATGTGTCC |
| | rs16889512 | 1.5947 | T | 68.809991 | AAGAAATGGCAAGTAA[C/T]GTTAAGAGACAAAATC |
| | rs17723785 | 1.7345 | G | 68.818409 | ACTGTTGCAGAGCTGA[C/G]TATGAAGAGATGACTC |
| | rs17780860 | 1.5090 | A | 68.832702 | AACTGAATGGTCTCTG[A/G]ATCTGGTAGCTAAAAG |
| Neuropathy | rs697076 | 1.2900 | A | 68.930656 | CTAGATATTCACGTGA[A/G]ATAGGATCATTCCACA |
| | rs294497 | 1.4600 | A | 68.941993 | TTATCTTTTCATTAGA[A/G]GGCAGGCCATTCTGAC |
| | rs294496 | 1.4500 | T | 68.942055 | CCATAAAGGATTTAGA[C/T]CAAAACAGAATAAAAC |
| | rs10514859 | 1.6000 | T | 68.942653 | GACCGATCAGAAGTTG[G/T]CAACAGGCCAGTGCTA |
| | rs294494 | 1.4800 | T | 68.943444 | GCCTCACAGTTAAGGA[C/T]ATTTTAGCTATTCAAG |
| | rs1035321 | 1.5800 | T | 68.943629 | CATCTGGTACCTGTCA[C/T]GGAATTTGATAATTCT |
| | rs36081664 | 1.6200 | A | 68.945118 | GCTTATATCATTGCCC[A/G]CAAATGGAAATGTAGC |
| | rs294492 | 1.238338 | A | 68.93421751 | TGAGAGGAACAGCATA[A/C]AAAATGATCCGCAAAA |
| | rs893190 | 1.5600 | T | 68.953457 | ATGGTGACGAATAGCA[C/T]CAATGATGAGTACACC |

### DMD: Dystrophin

### Chromosome: X

### Location: Xp21.2

TABLE 2
SNPs of DMD associated to 10 phenotypes with odd ratios and their positions

| | | | | Sex-averaged | |
|---|---|---|---|---|---|
| | | | Minor | Marshfield | |
| Phenotype | SNP ID | O.R. | Allele | position (cM) | Flanking sequence |
| Microvascular | rs2692986 | 1.231454 | G | 32.1633306 | CATGCATAAAGTGAAG[A/G]CTCCTTTCTGGGCTGA |
| disorder | rs17330097 | 1.5579 | T | 32.990042 | AAGGGCACCACGACTC[C/T]CTTGGAAAATTCTTTT |
| Micro_Macro- | rs5972687 | 1.241886 | A | 34.04788834 | TCAAATACATTTTAAC[A/G]ACTTCATCTTGTATAT |
| vascular | rs2692986 | 1.3788 | G | 32.163330 | CATGCATAAAGTGAAG[A/G]CTCCTTTCTGGGCTGA |
| disorder | | | | | |
| MACE | rs6527243 | 0.7317 | C | 34.136822 | TAGCATGGTGGCTTTA[A/C]GATACTCACACATTTT |
| Hypertension | rs1379106 | 0.840437 | T | 33.61451366 | CTGTTCTGCTAACATA[C/T]CCTCAATAAATCGATC |
| | rs5972470 | 0.83509 | T | 33.35819291 | TAGATAAAAGTCATAG[C/T]ACCTTACGTTGTAATT |
| | rs2692986 | 1.354934 | G | 32.1633306 | CATGCATAAAGTGAAG[A/G]CTCCTTTCTGGGCTGA |
| | rs10521991 | 0.8105 | C | 33.682701 | AGGTATCTGGATTTCA[C/T]CTGCCGGTTGTAAAAG |
| Atrial | rs5928032 | 1.6862 | A | 33.882164 | TCTAAATCATACATTG[A/G]AGGAAAATGGATAAAG |
| Fibrillation | rs5928033 | 1.6451 | A | 33.882254 | AGCAAATGTTAAAGAA[A/T]GGATGCAACTTATTTA |
| | rs808549 | 1.7412 | G | 33.882355 | GGTGACTATATCTCAG[G/T]CTCCCTCTTTGTAGTA |
| | rs5928038 | 1.5931 | A | 33.885514 | GTCAAAGGCATGATAG[A/G]CAGTATAGATTGCACA |
| | rs808521 | 1.7265 | T | 33.891106 | CATACAACTGCAGGTA[G/T]CTTTTTCATGTAACAC |
| | rs7884312 | 2.1063 | T | 34.049534 | TTGTAGCTTGACTGTC[C/T]CCAGCAAGAAAATGTC |
| | rs808517 | 1.384771 | A | 33.89279149 | AGGACTAGATTTAGGA[A/T]ATGAACAAAAAGCTCA |
| | rs7880606 | 1.9552 | T | 34.054635 | TAAATTAGGGATTTCC[A/T]GATAGGTGATGAAAAA |
| Neuropathy | rs6527237 | 0.8400 | A | 34.069333 | TTAGTGGGCAAAGAAG[A/G]ATAAGGGGTAAGCCAC |
| Low | rs5972687 | 1.135482 | A | 34.04788834 | TCAAATACATTTTAAC[A/G]ACTTCATCTTGTATAT |
| Creatinine | rs2692986 | 1.020221 | G | 32.1633306 | CATGCATAAAGTGAAG[A/G]CTCCTTTCTGGGCTGA |
| Clearance | | | | | |
| Retinopathy | rs5972687 | 1.038384 | G | 34.04788834 | TCAAATACATTTTAAC[A/G]ACTTCATCTTGTATAT |
| Albuminuria_ | rs2692986 | 1.277791 | G | 32.1633306 | CATGCATAAAGTGAAG[A/G]CTCCTTTCTGGGCTGA |
| Low | | | | | |
| Creatinine | | | | | |
| Clearance | | | | | |
| MACE_ | rs2692986 | 1.313732 | G | 32.1633306 | CATGCATAAAGTGAAG[A/G]CTCCTTTCTGGGCTGA |
| Albuminuria | | | | | |

SOX5: SRY (sex determining region Y)-box5

### Chromosome: 12

### Location: 12p12.1

TABLE 3
SNPs of SOX5 associated to 6 phenotypes with odd ratios and their positions

| | | | | Sex-averaged | |
|---|---|---|---|---|---|
| | | | Minor | Marshfield | |
| Phenotype | SNP ID | O.R. | Allele | position (cM) | Flanking sequence |
| Micro_Macro- | rs12426427 | 0.6966 | A | 43.142054 | ACAATACCTAGCTTAA[A/T]GCCTCAAACATAATAA |
| vascular | rs2728841 | 1.5038 | A | 43.568617 | TTAGATATCTTGGCAA[A/G]GGAAAAATGTATGTAA |
| disorder | | | | | |
| MI_Angina | rs2728841 | 1.4861 | A | 43.568617 | TTAGATATCTTGGCAA[A/G]GGAAAAATGTATGTAA |
| MACE | rs2728841 | 1.3286 | A | 43.568617 | TTAGATATCTTGGCAA[A/G]GGAAAAATGTATGTAA |
| MACE_ | rs12426427 | 0.7164 | A | 43.142054 | ACAATACCTAGCTTAA[A/T]GCCTCAAACATAATAA |
| Albuminuria | rs2728841 | 1.4139 | A | 43.568617 | TTAGATATCTTGGCAA[A/G]GGAAAAATGTATGTAA |
| Hypertension | rs7316665 | 1.5613 | C | 43.524727 | TTTCACTTGTGTTATC[C/T]AAATGCTTCCTACCTC |
| | rs2728841 | 1.3661 | A | 43.568617 | TTAGATATCTTGGCAA[A/G]GGAAAAATGTATGTAA |
| Atrial | rs16927597 | 1.5233 | G | 43.688981 | GTGTTTTGTGTTCATG[A/G]AAGAAAAAAACACACA |
| Fibrillation | rs16926892 | 0.681086 | G | 43.38355145 | TAAAACCTTCTGCAAT[A/G]AAAATTAACTTATTTA |
| | rs7963345 | 0.682177 | A | 43.38297666 | TCATACATGAGAGACA[A/T]ATAATAAGACCTGTTC |
| | rs4262802 | 1.6235 | C | 43.695317 | GCCGGTTAATGGATTA[C/T]GTGTATGAGTGGGTGG |

### SYT2: Synaptotagmin II

### Chromosome: 1

### Location: 1q32.1

TABLE 4
SNPs of SYT2 associated to 7 phenotypes with odd ratios and their positions

| | | | | Sex-averaged | |
|---|---|---|---|---|---|
| | | | Minor | Marshfield | |
| Phenotype | SNP ID | O.R. | Allele | position (cM) | Flanking sequence |
| Macrovascular | rs946857 | 1.3708 | A | 216.353413 | AACTGAGGTAAAGAGA[A/G]GTAAAATAACTTGTCC |
| disorder | rs12404969 | 1.3501 | T | 216.375803 | GATATATAGATTCACA[C/T]GCTTACCTAGAGCACC |
| | rs4537626 | 1.3348 | C | 216.395950 | ACATATGTGGAGGGCC[C/G]AACCTCGTGCCACAGC |
| | rs2153441 | 0.7028 | T | 216.412493 | TGCTGTGGAGTGACAC[C/T]GGGGGCTCTTTGCTGA |
| | rs7550433 | 0.7044 | G | 216.419342 | CCACTGATCACCATGT[C/G]AAAATCTCCTGGAGCA |
| | rs7517181 | 0.7028 | T | 216.419468 | GGTGATCAGTGGACCA[C/T]CTTTATTTTTTTATTT |
| | rs10920451 | 0.7375 | C | 216.462623 | ACTGTAAAACGTGCCA[C/T]ATGATAGTTTGAAAAC |
| | rs10920452 | 0.7342 | C | 216.462650 | CATTCAGTCCATAGCA[C/T]TGCATTTGTCAATTAT |
| | rs4950866 | 0.7394 | T | 216.463571 | TCATTCAGGCTTCTAA[C/T]GGCAAATGAGATCAGA |
| | rs2095981 | 0.7433 | G | 216.465368 | GGAACTGTGAGTTGAT[G/T]AAATCTCTCTTCTTCA |
| | rs4950867 | 0.7394 | C | 216.863682 | ATATACCTGTGCTCTC[C/T]CATTGTTCAGAAATTC |
| Micro_Macro- | rs946857 | 1.3336 | A | 216.353413 | AACTGAGGTAAAGAGA[A/G]GTAAAATAACTTGTCC |
| vascular | rs12404969 | 1.3268 | T | 216.375803 | GATATATAGATTCACA[C/T]GCTTACCTAGAGCACC |
| disorder | rs4537626 | 1.2683 | C | 216.395950 | ACATATGTGGAGGGCC[C/G]AACCTCGTGCCACAGC |
| | rs2153441 | 0.7907 | T | 216.412493 | TGCTGTGGAGTGACAC[C/T]GGGGGCTCTTTGCTGA |
| | rs7550433 | 0.7884 | G | 216.419342 | CCACTGATCACCATGT[C/G]AAAATCTCCTGGAGCA |
| | rs7517181 | 0.7616 | T | 216.419468 | GGTGATCAGTGGACCA[C/T]CTTTATTTTTTTATTT |
| MACE | rs946857 | 1.3593 | A | 216.353413 | AACTGAGGTAAAGAGA[A/G]GTAAAATAACTTGTCC |
| | rs12404969 | 1.3388 | T | 216.375803 | GATATATAGATTCACA[C/T]GCTTACCTAGAGCACC |
| | rs2153441 | 0.7212 | T | 216.412493 | TGCTGTGGAGTGACAC[C/T]GGGGGCTCTTTGCTGA |
| | rs7550433 | 0.7228 | G | 216.419342 | CCACTGATCACCATGT[C/G]AAAATCTCCTGGAGCA |
| | rs7517181 | 0.7208 | T | 216.419468 | GGTGATCAGTGGACCA[C/T]CTTTATTTTTTTATTT |
| MACE_ | rs2153441 | 0.8060 | T | 216.412493 | TGCTGTGGAGTGACAC[C/T]GGGGGCTCTTTGCTGA |
| Albuminuria | rs7550433 | 0.8040 | G | 216.419342 | CCACTGATCACCATGT[C/G]AAAATCTCCTGGAGCA |
| | rs7517181 | 0.8016 | T | 216.419468 | GGTGATCAGTGGACCA[C/T]CTTTATTTTTTTATTT |
| Hypertension | rs946857 | 1.3549 | A | 216.353413 | AACTGAGGTAAAGAGA[A/G]GTAAAATAACTTGTCC |
| | rs12404969 | 1.3609 | T | 216.375803 | GATATATAGATTCACA[C/T]GCTTACCTAGAGCACC |
| | rs4537626 | 1.3584 | C | 216.395950 | ACATATGTGGAGGGCC[C/G]AACCTCGTGCCACAGC |
| | rs2153441 | 0.7393 | T | 216.412493 | TGCTGTGGAGTGACAC[C/T]GGGGGCTCTTTGCTGA |
| | rs7550433 | 0.7334 | G | 216.419342 | CCACTGATCACCATGT[C/G]AAAATCTCCTGGAGCA |
| | rs7517181 | 0.7291 | T | 216.419468 | GGTGATCAGTGGACCA[C/T]CTTTATTTTTTTATTT |
| | rs4453099 | 1.3553 | T | 216.478408 | GTGTTCGGTCTGTCTG[C/T]AATACATTACTTTGAT |
| MI_Angina | rs946857 | 1.3858 | A | 216.353413 | AACTGAGGTAAAGAGA[A/G]GTAAAATAACTTGTCC |
| | rs12404969 | 1.3655 | T | 216.375803 | GATATATAGATTCACA[C/T]GCTTACCTAGAGCACC |
| | rs4537626 | 1.3420 | C | 216.395950 | ACATATGTGGAGGGCC[C/G]AACCTCGTGCCACAGC |
| | rs2153441 | 0.7091 | T | 216.412493 | TGCTGTGGAGTGACAC[C/T]GGGGGCTCTTTGCTGA |
| | rs7550433 | 0.7106 | G | 216.419342 | CCACTGATCACCATGT[C/G]AAAATCTCCTGGAGCA |
| | rs7517181 | 0.7084 | T | 216.419468 | GGTGATCAGTGGACCA[C/T]CTTTATTTTTTTATTT |
| Atrial | rs6698441 | 1.433952 | A | 216.4690538 | CCAAGCATGGCTTCTA[A/G]CACTATAGAAAAGGTT |
| Fibrillation | | | | | |

Polymorphic genes of the present invention comprise the genes/loci also disclosed in Tables 1-4. Particularly preferred are genes which are Phosphodiesterase 4D, cAMP-specific (PDE4D), Dystrophin (DMD), sex determining region Y (SRY)-box5 (SOX5), Synaptotagmin II (SYT2), or a combination thereof.

Oligonucleotide biomarkers of the instant invention include, but are not limited to, transcripts of the aforementioned genes. Such include primary transcripts and variants (e.g., processed, spliced, fragmented or derviatized primary mRNA) thereof, including complements thereof. For example, PDE4D biomarkers may comprise mRNAs having the sequence set forth in GENBANK accession Nos. NM_{—}001104631 (isoform 1), NM_{—}006203.4 (isoform 2), or NM_{—}001165899 (isoform 3). DMD biomarker may comprise mRNAs having the sequence set forth in GENBANK accession No. NM_{—}000109.3. SOX5 biomarkers may comprise mRNAs having the sequence set forth in GENBANK accession Nos. NM_{—}006940.4 (isoform a), NM_{—}152989.2 (isoform b), or NM_{—}178010.1 (isoform c). SYT2 biomarker may comprise mRNA having the sequence set forth in GENBANK accession Nos. NM_{—}177402.4 (variant 1) or NM_{—}001136504.1 (variant 2). These accessioned sequences are incorporated by reference in their entirety.

Protein biomarkers of the instant invention include, but are not limited to, polypeptide products of the aforementioned genes, including fragments thereof. Such include isoforms and variants of the wild-type protein. For example, PDE4D protein biomarkers may have the sequence set forth in GENBANK accession Nos. NP_{—}001098101 (isoform 1), NP_{—}006194 (isoform 2), NP_{—}001159371 ((isoform 3). DMD protein biomarker may comprise protein having the sequence set forth in GENBANK accession No. NP_{—}000100.2. Sox5 protein biomarkers may comprise proteins having the sequence set forth in GENBANK accession Nos. NP_{—}008871.3 (isoform a), NP_{—}694534.1 (isoform b), or NP_{—}821078.1 (isoform c). SYT2 protein biomarker may comprise protein having the sequence set forth in GENBANK accession Nos. NP_{—}796376.2 (variant 1) or NP_{—}001129976.1 (variant 2). These accessioned sequences are incorporated by reference in their entirety.

A “biomarker” can also be a clinical or biological biomarker. Clinical or biological biomarkers include, but are not limited to, age, sex, glucose levels, age of diagnosis, diabetes duration at baseline, cigarette smoking, diastolic or systolic blood pressure, atrial fibrillation, glycated hemoglobin (HbA1_{c}), total cholesterol, HDL cholesterol, albumin/creatinine ratio, glomerular filtration rate.

In one embodiment, the biomarker is one of the SNPs listed in Tables 1-4 or a SNP or a STR found to be in linkage disequilibrium to one of the SNP listed in Tables 1-4.

In another embodiment, the biomarker is a SNP of at least one of the genes listed in Tables 1-4 or a STR linked to a SNP of at least one of these above genes or to a locus closely related thereto.

The present invention thus provides for methods of predicting risk of complications associated with T2D, comprising detecting at least one of the SNPs listed in Tables 1-4 or a SNP or a STR found to be in linkage disequilibrium with one or more of the SNPs listed in Tables 1-4, or a SNP of at least one gene listed in Tables 1-4 or a SNP or a STR found to be in linkage disequilibrium with a SNP of such a gene, wherein the presence of the SNP or STR in a sample of a subject (or patient) suffering from T2D indicates that said subject (or patient) is likely to develop the complication. Preferred examples of such complications include, but are not limited to, micro/macrovascular disorder, hypertension, neuropathy, atrial fibrillation, nephropathy, or MACE.

As used herein, “single nucleotide polymorphism,” or “SNP” is a DNA sequence variation that occurs when a nucleotide, e.g., adenine (A), thymine (T), cytosine (C), or guanine (G), in the genome sequence is altered to another nucleotide. SNPs are occasional variations in DNA sequence; the vast majority of the DNA sequence is identical among all humans. SNPs or other variants may also be found in genomic regions that do not contain genes. They represent a genomic hot spot responsible for the genetic variability among humans.

The SNPs of the instant invention are those having the RefSNPIDs listed in Tables 1-4. Such include, but are not limited to, SNPs having the refSNPIDs (RS) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441. Combinations of SNPs may also be detected.

As used herein, “gene” means any amount of nucleic acid material that is sufficient to encode a transcript or protein having the function desired. Thus, it includes, but is not limited to, genomic DNA, cDNA, RNA, and nucleic acid that are otherwise genetically engineered to achieve a desired level of expression under desired conditions. Accordingly, it includes fusion genes (encoding fusion proteins), intact genomic genes, and DNA sequences fused to heterologous promoters, operators, enhancers, and/or other transcription regulating sequences. Methods and nucleic acid constructs for preparing genes for recombinant expression are well known and widely used by those of skill in the art, and thus need not be detailed here. The term refers to an entirety containing entire transcribed region and all regulatory regions of a gene. The transcribed region of a gene including all exon and intron sequences of a gene including alternatively spliced exons and introns so the transcribed region of a gene contains in addition to polypeptide encoding region of a gene also regulatory and 5′ and 3′ untranslated regions present in transcribed RNA.

Exemplary genes of the invention are listed in Tables 1-4. Such include, but are not limited to, Phosphodiesterase 4D, cAMP-specific (PDE4D; NCBI Gene ID: 5144), Dystrophin (DMD; NCBI Gene ID: 1756), sex determining region Y (SRY)-box5 (50×5; NCBI Gene ID: 6660), Synaptotagmin II (SYT2; NCBI Gene ID: 127833). Combinations of genes may also be detected. The sequences of these genes, including sequences of the primary transcripts (mRNA) and protein products encoded thereby are incorporated by reference herein in their entirety.

As used herein, an “exon” is a segment of a eukaryotic gene that encodes a sequence of nucleotides in mRNA. An exon can encode amino acids in a protein. Exons are generally adjacent to introns.

As used herein, an “intron” is a non-coding region of a eukaryotic gene that may be transcribed into an RNA molecule, but is not usually translated into amino acids. It may be excised by RNA splicing when mRNA is produced.

As used herein, a “patient” is any living animal, including, but not limited to, a human who has, or is suspected of having or being susceptible to, a disease or disorder, or who otherwise would be a subject of investigation relevant to a disease or disorder. Accordingly, a patient can be an animal that has been bred or engineered as a model for metabolic syndrome, type 2 diabetes, obesity, hypertension, atherosclerosis, or any other disease or disorder. Likewise it can be a human suffering from, or at risk of developing, a disease or disorder associated with insulin metabolism, including but not limited to type 2 diabetes, or any other disease or disorder. Similarly, a patient can be an animal (such as an experimental animal, a pet animal, a farm animal, a dairy animal, a ranch animal, or an animal cultivated for food or other commercial use), or a human, serving as a healthy control for investigations into diseases and/or disorders, e.g., those associated with insulin metabolism.

By “reagent,” is meant any element, molecule, or compound that is present in the assay system and participates, either directly or indirectly, in the biochemical processes occurring during the performance of the method. Reagents include, but are not limited to, nucleic acids, cells, media, chemicals, compounds used to introduce nucleic acids into cells, and compounds used to generate detectable signals.

By “materials” it is meant items that are used to contain and/or perform the methods of the invention, but that do not participate in any of the biochemical reactions taking place in the method. Materials include, but are not limited to, test tubes, pipettes, gels, and ultraviolet transilluminators.

A “haplotype,” as described herein, refers to any combination of genetic markers (“alleles”) usually inherited together. A haplotype can comprise two or more alleles and the length of a genome region comprising a haplotype may vary from few hundred bases up to hundreds of kilobases. As it is recognized by those skilled in the art, the same haplotype can be described differently by determining the haplotype defining alleles from different nucleic acid strands. For example, the haplotype GGC defined by the SNP markers of this invention is the same as haplotype CCG in which the alleles are determined from the other strand, or haplotype CGC, in which the first allele is determined from the other strand. The haplotypes described herein are differentially present in T2D patients with increased risk of developing one or more of the aforementioned complications. Therefore, these haplotypes have diagnostic value for risk assessment, diagnosis and prognosis of T2D-related complications. Detection of haplotypes can be accomplished by methods known in the art used for detecting nucleotides at polymorphic sites.

A nucleotide position in genome at which more than one sequence is possible in a population, is referred to herein as a “polymorphic site” or “polymorphism”. Where a polymorphic site is a single nucleotide in length, the site is referred to as a SNP. For example, if at a particular chromosomal location, one member of a population has an adenine and another member of the population has a cytosine at the same position of his or her paternal or maternal DNA molecule, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a cytosine allele. Typically, a reference nucleotide sequence is referred to for a particular polymorphism e.g. in NCBI databases (as accessible on the world-wide-web at ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences. Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by the genes comprising such SNPs. For example, a nucleotide change resulting in a change in polypeptide sequence can alter the physiological properties of a polypeptide dramatically by resulting in altered activity, distribution and stability or otherwise affect on properties of a polypeptide. Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of a susceptibility gene, such as, for example, an SNP associated with the aforementioned genes.

The SNP markers of the present invention which are disclosed in Tables 1-4 have been denoted with their official reference SNP (rs) ID identification tags assigned to each unique SNP by the National Center for Biotechnological Information (NCBI). Each rs ID has been linked to specific variable alleles present in a specific nucleotide position in the human genome, and the nucleotide position has been specified with the nucleotide sequences flanking each SNP.

Although the numerical chromosomal position of a SNP may still change upon annotating the current human genome build, the SNP identification information such as variable alleles and flanking nucleotide sequences assigned to a SNP will remain the same. Those skilled in the art will readily recognize that the analysis of the nucleotides present in one or more SNPs set forth in Tables 1-4 of this invention in an individual's nucleic acid can be done by any method or technique capable of determining nucleotides present in a polymorphic site using the sequence information assigned in prior art to the rs IDs of the SNPs listed in Tables 1-4 of this invention. As it is obvious in the art the nucleotides present in polymorphisms can be determined from either nucleic acid strand or from both strands.

In one embodiment, the invention relates to a method for predicting the risk of developing a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine Clearance or nephropathy in a subject having T2D, comprising detecting in a sample obtained from said subject at least one SNP having an RefSNP ID listed in Tables 1-4.

Examples of such SNPs include, but are not limited to, for example, rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099, and rs6698441.

As described in the Brief Description of the Tables section of this application, Table 1 relates to the SNPs of PDE4D. These include, but are not limited to, for example, SNPs with RefSNPID rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190. SNPs with RefSNPID rs12657171 and rs11951359 were linked to micro and micro/macrovascular disorders, MACE and albuminuria. SNP with RefSNPID rs11951359 was associated with MACE and hypertension. SNPs with RefSNPID rs294492 and rs294494 were associated with atrial fibrillation. SNP with RefSNPID rs16889508 was associated with neuropathy.

Similarly, Table 2 relates to the SNPs of DMD. These include but are not limited to SNPs with RefSNPID rs17330097, rs5972687, rs2692986, rs6527243, rs1379106, rs5972470, rs10521991, rs5928032, rs5928033, rs808549, rs5928038, rs808521, rs7884312, rs808517, rs7880606 and rs6527237. Herein, SNP with RefSNPID rs16889508 was associated with atrial fibrillation and hypertension. SNPs with RefSNPID rs808517 and rs808521 were also associated with atrial fibrillation, while SNP with RefSNPID rs5972470 and rs2692986 were also associated with hypertension. SNP with RefSNPID rs5972687 and rs2692986 were associated with micro and micro/macrovascular disorders.

Table 3 relates to the SNPs of SOX5. These include, but are not limited to, for example, SNPs with RefSNPID rs12426427, rs2728841, rs7316665, rs16927597, rs16926892, rs7963345 and rs4262802. Herein, SNP with RefSNPID rs2728841 was found to be linked to both macro/micro vascular disorders and hypertension. SNPs with RefSNPID rs16927597, rs16926892 and rs7963345 were linked to atrial fibrillation.

Table 4 relates to the SNPs of SYT2. These include, but are not limited to, for example, SNPs with RefSNPID rs946857, rs12404969, rs4537626, rs2153441, rs7550433, rs7517181, rs10920451, rs10920452, rs4950866, rs2095981, rs4950867, rs4453099 and rs6698441. Herein, SNP with RefSNPID rs4537626 was found to be linked to both macro/micro vascular disorders and hypertension. SNPs with RefSNPID rs7517181 and rs12404969 were found to be singularly associated with hypertension. SNPs with RefSNPID rs7517181 and rs12404969 were found to be singularly associated with micro-macro vascular complications. SNP with RefSNPID rs6698441 was found to be linked to atrial fibrillation.

The following SNPs (and the genes linked therewith) are associated with specific T2D-related complications:

Hypertension: rs829258, rs10051847, rs11951359, rs10461656 (PDE4D); rs1379106, rs5972470, rs2692986, rs10521991 (DMD); rs7316665, rs2728841 (SOX5); rs946857, rs12404969, rs4537626, rs2153441, rs7550433, rs7517181, rs4453099 (SYT2)

Micro-macro vascular disorders: rs10051847, rs11951359, rs12657171, rs1077183, rs10514870, rs10066573, rs10068543, rs6450502, rs6864156, rs33927508, rs16889615, rs10471476 (PDE4D); rs5972687, rs2692986 (DMD); rs12426427, rs2728841 (SOX5); rs946857, rs12404969, rs4537626, rs2153441, rs7550433, rs7517181 (SYT2).

MACE: rs10051847, rs11951359, rs12657171, rs1077183 (PDE4D); rs6527243 (DMD); rs2728841 (SOX5); rs946857, rs12404969, rs2153441, rs7550433, rs7517181 (SYT2)

MACE+albuminuria: rs829258, rs10051847, rs11951359, rs10461656, rs12657171, rs1077183, rs10514870, rs10066573, rs10068543, rs6450502, rs6864156 (PDE4D); rs2692986 (DMD); rs12426427, rs2728841 (SOX5); rs2153441, rs7550433, rs7517181 (SYT2).

Atrial fibrillation: rs16889508, rs17528550, rs16889512, rs17723785, rs17780860 (PDE4D); rs5928032, rs5928033, rs808549, rs5928038, rs808521, rs7884312, rs808517, rs7880606 (DMD); rs16927597, rs16926892, rs7963345, rs4262802 (SOX5); rs6698441 (SYT2)

Neuropathy: rs697076, rs294497, rs294496, rs10514859, rs294494, rs1035321, rs36081664, rs294492, rs893190 (PDE4D); rs6527237 (DMD).

The following provides a list of SNPs that are associated with one or more complications:

SNPs Grouped by Complication

Have albuminuria: rs10051847 (PDE4D)

Macrovascular disorder: rs10051847, rs11951359, rs2968005 (PDE4D); rs946857, rs12404969, rs4537626, rs2153441, rs7550433, rs7517181, rs10920451, rs10920452, rs4950866, rs2095981, rs4950867 (SYT2)

Micro_Macrovascular disorder: rs10051847, rs11951359, rs12657171, rs1077183, rs10514870, rs10066573, rs10068543, rs6450502, rs6864156, rs33927508, rs16889615, rs10471476 (PDE4D); rs5972687, rs2692986 (DMD); rs12426427, rs2728841 (SOX5); rs946857, rs12404969, rs4537626, rs2153441, rs7550433, rs7517181 (SYT2).

MI_Angina: rs10051847, rs11951359, rs12657171, rs1077183 (PDE4D); rs2728841 (SOX5); rs946857, rs12404969, rs4537626, rs2153441, rs7550433, rs7517181 (SYT2)

MACE: rs10051847, rs11951359, rs12657171, rs1077183 (PDE4D); rs6527243 (DMD); rs2728841 (SOX5); rs946857, rs12404969, rs2153441, rs7550433, rs7517181 (SYT2)

MACE_Albuminuria: rs829258, rs10051847, rs11951359, rs10461656, rs12657171, rs1077183, rs10514870, rs10066573, rs10068543, rs6450502, rs6864156 (PDE4D); rs2692986 (DMD); rs12426427, rs2728841 (SOX5); rs2153441, rs7550433, rs7517181 (SYT2)

Hypertension: rs829258, rs10051847, rs11951359, rs10461656 (PDE4D); rs1379106, rs5972470, rs2692986, rs10521991 (DMD); rs7316665, rs2728841 (SOX5); rs946857, rs12404969, rs4537626, rs2153441, rs7550433, rs7517181, rs4453099 (SYT2).

Atrial fibrillation: rs16889508, rs17528550, rs16889512, rs17723785, rs17780860 (PDE4D); rs5928032, rs5928033, rs808549, rs5928038, rs808521, rs7884312, rs808517, rs7880606 (DMD); rs16927597, rs16926892, rs7963345, rs4262802 (SOX5); rs6698441 (SYT2).

Neuropathy: rs697076, rs294497, rs294496, rs10514859, rs294494, rs1035321, rs36081664, rs294492, rs893190 (PDE4D); rs6527237 (DMD).

Microvascular disorder: rs2692986, rs17330097 (DMD)

Low Creatinine Clearance: rs5972687, rs2692986 (DMD)

Retinopathy: rs5972687 (DMD)

Albuminuria_Low Creatinine Clearance: rs2692986 (DMD)

Without listing each and every possible combination, it is contemplated that one skilled in the art can readily identify the genes associated with the aforementioned SNPs and associate said genes with the aforementioned disorders.

Preferably, the invention relates to a method for predicting the risk of developing a complication which is micro/macrovascular disorder, hypertension, atrial fibrillation, or neuropathy in a subject having T2D, comprising detecting in a sample obtained from said subject at least one SNP having an RefSNP ID listed in Table 1. These SNPs are rs11951359, rs12657171, rs16889508, rs294492, and rs294494 and all of them are within introns. rs11951359 and rs12657171 were associated significantly (in most cases p<10⁻³) to micro and micro/macrovascular disease or MACE+albuminuria; rs11951359 to hypertension or MACE; rs16889508 to AF; rs294492 and rs294494 to neuropathy (Table 1).

In a related aspect, the present invention relates to a method for predicting the risk of developing a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject having T2D, comprising detecting at least one gene having a SNP listed in Tables 1-4.

Preferably, the invention relates to a method for predicting the risk of developing a complication which is micro/macrovascular disorder, hypertension, atrial fibrillation, or neuropathy in a subject having T2D, comprising detecting in a sample obtained from said subject at least one gene listed in Table 1. This gene is Phosphodiesterase 4D, cAMP-specific (PDE4D; NCBI Gene ID: 5144)

Determination of Repeated Sequences

The present invention also provides a method for prognosticating T2D-related complication in a subject comprising detecting short tandem repeats (STR) in linkage disequilibrium with a SNP listed in Table 1-4. The present invention thus provides for methods of predicting risk of complication associated with T2D, comprising detecting at least one STR found to be in linkage disequilibrium with one of the SNPs of the present invention, wherein the presence of the STR in a sample of a subject (or patient) suffering from T2D indicates that said subject (or patient) is likely to develop the complication. Preferred examples of such complications include, but are not limited to, micro/macrovascular disorder, hypertension, neuropathy, atrial fibrillation, nephropathy or MACE. Methods for determining the presence of repeated sequences in a nucleic acid sample (for example, genomic DNA) are known in the art.

As such, in a related embodiment, the present invention provides a method for prognosticating type 2 diabetes-related complications in a subject comprising detecting single tandem repeats (STR) in a nucleic acid target sequence, wherein such target sequences are contained in at least one gene from the aforementioned gene set or a locus related thereto. The nucleotide sequences contained in the genes and/or a locus related thereto are obtainable from the GENEID and/or OMIM accession numbers.

It is understood that the SNP markers of this invention may be associated with other polymorphisms. This allows for tagging SNPs (tagSNPs), which comprise loci that can serve as proxies for many other SNPs. The use of tagSNPs greatly improves the power of association studies as only a subset of loci needs to be genotyped while maintaining the same information and power as if one had genotyped a larger number of SNPs.

By using the name of the aforementioned gene provided in Tables 1-4 those skilled in the art will readily find the nucleotide sequences of a gene and the mRNAs encoded thereby as well as amino acid sequences the encoded polypeptides.

In certain methods described herein, an individual who is at risk for a T2D-related complication is an individual in whom one or more SNPs selected from the Tables 1-4 are identified. In other embodiment also polymorphisms or haplotypes associated to SNPs of the tables may be used in risk assessment of a T2D-related complication. The significance associated with an allele or a haplotype is measured by an odds ratio. In a further embodiment, the significance is measured by a percentage. In one embodiment, a significant risk is measured as odds ratio of 0.9 or less or at least about 1.1, including by not limited to: 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 10%, including but not limited to about 10%, 25%, 30%, 35%, 40%, 45%, 50%, 55%, 60%, 65%, 70%, 75%, 80%, 85%, 90%, 95% and 99%. In a further embodiment, a significant increase in risk is at least about 50%. It is understood however, that identifying whether a risk is medically significant may also depend on a variety of factors such as family history of hypertension, history of gestational diabetes, previously identified glucose intolerance, obesity, hypertriglyceridemia, hypercholesterolemia, elevated LDL cholesterol, low HDL cholesterol, elevated blood pressure (BP), cigarette smoking, lack of physical activity, and inflammatory components as reflected by increased C-reactive protein levels or other inflammatory markers.

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize to its specific target. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Probes and primers may include modified bases as in polypeptide nucleic acids. Probes or primers typically comprise about 15 to 30 consecutive nucleotides present e.g. in human genome and they may further comprise a detectable label, e.g., radioisotope, fluorescent compound, enzyme, or enzyme co-factor. Probes and primers to a SNP marker disclosed in Tables 1-4 are available in the art or can easily be designed using the flanking nucleotide sequences assigned to a SNP rs ID and standard probe and primer design tools.

Primers and probes for SNP markers disclosed in Tables 1-4 can be used in risk assessment as well as molecular diagnostic methods and kits of this invention.

The invention comprises polyclonal and monoclonal antibodies that bind to a polypeptide encoded by a gene listed in table 1-4 or comprising a SNP set forth in Tables 1-4 of the invention. Preferably, the antibodies of the instant invention bind to PDE4D protein biomarkers having the sequence set forth in GENBANK accession Nos. NP_{—}001098101 (isoform 1), NP_{—}006194 (isoform 2), NP_{—}001159371 ((isoform 3) or bind to DMd protein biomarker comprising the sequence set forth in GENBANK accession No. NP_{—}000100.2 or bind to Sox5 protein biomarkers having the sequence set forth in GENBANK accession Nos. NP_{—}008871.3 (isoform a), NP_{—}694534.1 (isoform b), or NP_{—}821078.1 (isoform c) or bind to Syt2 protein biomarker having the sequence set forth in GENBANK accession Nos. NP_{—}796376.2 (variant 1) and NP_{—}001129976.1 (variant 2).

The term “antibody” as used herein refers to immunoglobulin molecules or their immunologically active portions that specifically bind to an epitope (antigen, antigenic determinant) present in a polypeptide or a fragment thereof, but does not substantially bind other molecules in a sample, e.g., a biological sample, which contains the polypeptide. Examples of immunologically active portions of immunoglobulin molecules include F(ab) and F(ab′)₂ fragments which can be generated by treating the antibody with an enzyme such as pepsin. The term “monoclonal antibody” as used herein refers to a population of antibody molecules that are directed against a specific epitope and are produced either by a single clone of B cells or a single hybridoma cell line. Polyclonal and monoclonal antibodies can be prepared by various methods known in the art. Additionally, recombinant antibodies, such as chimeric and humanized monoclonal antibodies, comprising both human and non-human portions, can be produced by recombinant DNA techniques known in the art. Antibodies can be coupled to various enzymes, prosthetic groups, fluorescent materials, luminescent materials, bioluminescent materials, or radioactive materials to enhance detection.

In a related embodiment, the present invention also provides for the use of antisense oligonucleotides or silencing RNAs or similar methods which are capable of modulating the expression and/or levels of a product (i.e., mRNA or polypeptide) of a gene comprising a SNP set forth in Table 1-4. In a particularly preferred embodiment, the antisense molecules silencing RNAs or similar methods of the present invention are useful directed against the primary transcript (i.e., mRNA) of the genes listed in Tables 1-4. Techniques for the design and use of antisense molecules or silencing RNAs or similar methods, for example, in in vitro and/or in vivo applications, are known in the art.

“A T2D-related complication” in the context of this invention refers to glucose intolerance, insulin resistance, metabolic syndrome, obesity, a microvascular complication of T2D such as retinopathy, nephropathy or neuropathy, or a micro/macrovascular complication such as coronary heart disease, cerebrovascular disease, congestive heart failure, claudication or other clinical manifestation of atherosclerosis or arteriosclerosis. Preferred complications include, for example, micro/macrovascular disorder, hypertension, neuropathy, atrial fibrillation, nephropathy or MACE.

Particularly preferred types of “T2D-related complications” include, but are not limited to, micro/macrovascular disorder, hypertension, neuropathy, atrial fibrillation and MACE.

An antibody specific for a polypeptide encoded by a gene identified in Tables 1-4 of the invention can be used to detect the polypeptide in a biological sample in order to evaluate the abundance and pattern of expression of the polypeptide. Antibodies can be used diagnostically to monitor protein levels in tissue such as blood as part of a test predicting the susceptibility to complications, such as, for example, micro/macrovascular disorder, hypertension, neuropathy, atrial fibrillation, nephropathy or MACE. Antibodies may also be used as part of a clinical testing procedure, e.g., to, for example, determine the efficacy of a given treatment regimen. Highly purified antibodies (e.g. monoclonal humanized antibodies specific to a polypeptide encoded by an associated gene of the invention and/or polymorphic gene may be produced using GMP-compliant manufacturing processes known in the art. These “pharmaceutical grade” antibodies can be used in novel therapies modulating activity and/or function of a polypeptide encoded the associated gene(s) disclosed herein.

This invention provides information on genomic markers that can be used to develop methods, reagents and kits useful to predict diabetes complications. Development of such methods, reagents and kits relies on methods known to those skilled in the art, including without limitation allele specific PCR amplification or detection of such alleles, with or without prior amplification, with allele specific probes, and DNA sequencing. Information on genomic DNA sequences from which PCR primers, hybridization probes, and sequencing primers can designed can be found in public databases using the rs ID provided for each SNP in Tables 1-4.

Diagnostic Methods and Test Kits

One major application of the current invention is diagnosing a susceptibility to T2D related complications. The risk assessment methods and test kits of this invention can be applied to any diabetic patient as a screening or predisposition test, although the methods and test kits are also be applied to prediabetic patients and other subjects, preferably those with high-risk individuals (who have e.g. family history of T2D, history of gestational diabetes, previous glucose intolerance, obesity or any combination of these). Diagnostic tests that define genetic factors contributing to T2D complications might be used together with or independent of the known clinical risk factors to define an individual's risk relative to the general population. Better means for identifying those individuals susceptible for a T2D-related complication should lead to better preventive and treatment regimens, including more aggressive management of the risk factors for a T2D-related complication such as obesity, lack of physical activity, hypercholesterolemia, elevated LDL cholesterol, low HDL cholesterol, elevated BP, cigarette smoking and inflammatory components as reflected by increased C-reactive protein levels or other inflammatory markers. Physicians may use the information on genetic risk factors to convince particular patients to adjust their life style e.g. to stop smoking, to reduce caloric intake or to increase exercise.

In one embodiment of the invention, diagnosis of a susceptibility to T2D related complication in a subject is made by detecting one or more SNP markers disclosed in Tables 1-4 of this invention in the subject's nucleic acid. The presence of assessed SNP markers or haplotypes in individual's genome indicates subject's increased risk for said T2D related complication. The invention also pertains to methods of diagnosing a susceptibility to said complication in an individual comprising detection of a haplotype in a genetic aspect that is more frequently present in an individual having a T2D complication (affected), compared to the frequency of its presence an individual not having a T2D complication (control), wherein the presence of the haplotype is indicative of a susceptibility to T2D-related complication. A haplotype may be associated with a reduced rather than increased risk of said complication, wherein the presence of the haplotype is indicative of a reduced risk of T2D-related complication.

In other embodiment of the invention, diagnosis of susceptibility to T2D-related complication, is done by detecting in the subject's nucleic acid one or more polymorphic sites which are in linkage disequilibrium with one or more SNP markers disclosed in Tables 1-4 of this invention. For a therapeutic purpose, the most useful polymorphic sites are those altering the biological activity of a polypeptide encoded by a T2D related complication gene set forth in Tables 1-4. Examples of such functional polymorphisms include, but are not limited to frame shifts; premature stop codons, amino acid changing polymorphisms and polymorphisms inducing abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other useful polymorphic sites are those affecting transcription of a gene set forth in Tables 1-4 or comprising a SNP associated thereto. Such polymorphisms may affect the translation of mRNA due to altered tissue specificity, due to altered transcription rate, due to altered response to physiological status, due to altered translation efficiency of the mRNA and due to altered stability of the mRNA. The presence of nucleotide sequence variants altering the polypeptide structure and/or expression in said associated genes in individual's nucleic acid is diagnostic for susceptibility to T2D-related complication but for a diagnostic purpose, the variant may also be included in uncharted areas of the genome.

In diagnostic assays determination of the nucleotides present in one or more SNP markers of this invention, as well as polymorphic sites associated therewith can be done by any method or technique which can accurately determine nucleotides present in a polymorphic site. Numerous suitable methods have been described in the art. These methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is evident in the art, the nucleotides present in a polymorphic site can be determined from either nucleic acid strand or from both strands.

In another embodiment of the invention, a susceptibility to a T2D-related complication is assessed from transcription products of one or more associated genes. Qualitative or quantitative alterations in transcription products can be assessed by a variety of methods described in the art, including e.g. hybridization methods, enzymatic cleavage assays, RT-PCR assays and microarrays. A test sample from an individual is collected and the alterations in the transcription of associated genes are assessed from the RNA molecules present in the sample. Altered transcription is diagnostic for a susceptibility to a T2D-related complication.

In another embodiment of the invention, diagnosis of a susceptibility to T2D-related complication is made by examining expression, abundance, biological activities, structures and/or functions of polypeptides encoded by one of the gene disclosed in Tables 1-4. A test sample from an individual is assessed for the presence of alterations in the expression, biological activities, structures and/or functions of the polypeptides, or for the presence of a particular polypeptide variant (e.g., an isoform) encoded by a gene disclosed in Tables 1-4. An alteration can be, for example, quantitative (an alteration in the quantity of the expressed polypeptide, i.e., the amount of polypeptide produced) or qualitative (an alteration in the structure and/or function of a polypeptide encoded by the polymorphic genes could be measured. Alterations in expression, abundance, biological activity, structure and/or function of polypeptides encoded by such polymorphic genes can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the polypeptide itself or its fragment or from substrates and reaction products of said polypeptide. A control sample is a sample that corresponds to the test sample (e.g., is from the same type of cells), and is from an individual who is not affected by a T2D complication. An alteration in the expression, abundance, biological activity, function or composition of a polypeptide encoded by a polymorphic gene of the invention in the test sample, as compared with the control sample, is indicative of a susceptibility to developing complications. In another embodiment, assessment of the splicing variant or isoform(s) of a polypeptide encoded by a polymorphic gene can be performed directly (e.g., by examining the polypeptide itself), or indirectly (e.g., by examining the mRNA encoding the polypeptide, such as through mRNA profiling).

Yet in another embodiment, a susceptibility to a T2D-related complication can be diagnosed by assessing the status and/or function of biological networks and/or metabolic pathways related to one or more polypeptides encoded by a T2D-related complication risk gene of this invention. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop said complication is evaluated by comparing observed status and/or function of biological networks and or metabolic pathways of a subject to the status and/or function of biological networks and or metabolic pathways of healthy controls.

Another major application of the current invention is diagnosis of a molecular subtype of a type II diabetic patient. Molecular diagnosis methods and kits of this invention can be applied to a person having type II diabetes. In one preferred embodiment, molecular subtype of T2D in an individual is determined to provide information of the molecular etiology of T2D. When the molecular etiology is known, better diagnosis and prognosis of T2D can be made and efficient and safe therapy for treating T2D-related complications in an individual can be selected on the basis of this genetic subtype. For example, a drug that is likely to be effective, for example, a blood glucose lowering agent, can be selected without trial and error. Physicians may use the information on genetic risk factors with or without known clinical risk factors to convince particular patients to adjust their life style and manage T2D risk factors and select intensified preventive and curative interventions for them. In other embodiment, biomarker information obtained from methods and kits of the present invention are used to select human subjects for clinical trials testing anti-diabetic drugs. The kits provided for diagnosing a molecular subtype of T2D in an individual comprise wholly or in part protocol and reagents for detecting one or more biomarkers and interpretation software for data analysis and T2D molecular subtype assessment.

The diagnostic assays and kits of the invention may further comprise a step of combining non-genetic information with the biomarker data to make risk assessment, diagnosis or prognosis of a T2D-related complication. Useful non-genetic information comprises, without limitations, age, gender, smoking status, physical activity, waist-to-hip circumference ratio (cm/cm), the subject family history of T2D or obesity, history of gestational diabetes, previously identified glucose intolerance, obesity, hypertriglyceridemia, low HDL cholesterol, HT and particularly elevated BP and/or status of being hypertensive. The detection method of the invention may also further comprise a step determining blood, serum or plasma glucose, total cholesterol, HDL cholesterol, LDL cholesterol, triglyceride, apolipoprotein B and AI, fibrinogen, ferritin, transferrin receptor, C-reactive protein, serum or plasma insulin concentration.

The score that predicts the probability of developing a T2D-related complication may be calculated using art-known procedures including but not limited to logistic regression, support vector machines and neural networks. The results from the further steps of the method as described below render possible a step of calculating the probability of developing such T2D-related complication using a logistic regression equation. Alternative statistical models include, but are not limited to, Cox's proportional hazards' model, other iterative models and neural networking models.

Diagnostic test kits (e.g. reagent kits) of this invention comprise reagents, materials and protocols for assessing one or more biomarkers, and instructions and software for comparing the biomarker data from a subject to biomarker data from healthy and diseased people to make risk assessment, diagnosis or prognosis of a T2D related complication and optimized therapeutic suggestions. Useful reagents and materials for kits include, but are not limited to PCR primers, hybridization probes and primers as described herein (e.g., labeled probes or primers), allele-specific oligonucleotides, reagents for genotyping SNP markers, reagents for detection of labeled molecules, restriction enzymes (e.g., for RFLP analysis), DNA polymerases, RNA polymerases, DNA ligases, marker enzymes, antibodies which bind to altered or to non-altered (native) a polypeptide, means for amplification of nucleic acids fragments from one or more SNPs selected from the Tables 1-4, means for analyzing the nucleic acid sequence of one or more T2D-complication related SNPs, or means for analyzing the sequence of one or more amino acid residues of polypeptides encoded by genes comprising such SNPs, etc. In one embodiment, a kit for diagnosing susceptibility to a T2D-related complication comprises primers and reagents for detecting the nucleotides present in one or more SNP markers selected from the Tables 1-4 in individual's nucleic acid.

Selection of Patients for Clinical Trials

Diabetes is very commonly associated with a significant risk of subsequent complications, such as cardiovascular diseases, stroke, micro/macrovascular complications, and/or microvascular complications. Health Authorities, especially the US Food and Drug Administration (FDA), are concerned about recent reports of an increased rate of cardiovascular complications associated with the use of some anti-diabetic drugs. In order to protect the population, the FDA has requested very costly clinical studies to evaluate the cardiovascular risk of new diabetes drugs. For instance, in July 2008, the FDA convened a two-day meeting to discuss whether morbidity/mortality cardiovascular outcomes trials should be part of the approval process for pharmacological therapies developed for T2D. The resulting guidance was issued in December 2008 (Guidance for Industry: Diabetes Mellitus—Evaluating cardiovascular risk in new antidiabetic therapy to treat type 2 diabetes). In summary, the current standard for evaluation of efficacy have been strengthened by requiring further safety assessments, including long term (minimum 2 years) cardiovascular evaluation, based either on meta-analysis or an ad hoc clinical trial with sufficient “hard” cardiovascular outcomes in the control arm. In this document the FDA insists on recruiting patients with higher risk of cardiovascular events. Further guidelines are expected soon for drugs that have already been approved. The new requirements may, in turn, lead to the discontinuation of new drug development, and redirect available funding for diabetes research to other conditions.

One way to improve outcome is to properly stratify patients for risk of adverse side effects. The current methods for selecting patient populations for such studies are based on well-established risk factors, such as previous cardiovascular events, unfavorable lipid or blood pressure profile, increased CRP, etc. These approaches are partially effective since clinical trials would require upwards of 50 000 subjects if unselected T2D patients were randomized. The use of clinical and biological biomarker-based characterization has reduced the pool size to about 15,000. Nonetheless, this still remains one of the most costly (more than US$150M) and time consuming steps in drug development. An example is a trial launched in November 2008 by Merck, Inc. on the cardiovascular safety of sitagliptin (JANUVIA) (NCT 00790205) in T2D subjects. This trial required 14,000 patients, even though pre-existing cardiovascular disease was utilized as a criterion for inclusion in this study.

Correlations between genomic signatures as defined by SNPs and/or STRs combined with clinical/biological biomarkers, and cardiovascular complications in T2D patients provide a way to improve a researcher's ability to perform clinical outcome studies in a population. Using the methods and compositions in accordance with the embodiments of the instant invention herein disclosed, it is possible to reduce dramatically the number of subjects, and hence the cost of clinical studies, by selecting a filtered patient cohort comprising, for example, high-risk patients that are more likely to develop more cardiovascular complications than the general population of diabetes patients. Hence, in an embodiment of the instant invention, there is provided a means for identifying relevant genetic information and combining such information with other patient characteristics, such as, for example, age, sex, duration of diabetes, glycated hemoglobin, LDL and HDL cholesterol, hypertension, smoking, atrial fibrillation, ankle-arm blood pressure indices, pulse, symptomatic claudication and/or albuminuria, etc., which will be made available to companies developing new anti-diabetic drugs. Using the methods and compositions of the present invention, a researcher/clinician can identify a suitable patient cohort. Such may include, for example, patients likely to develop one or more of the aforementioned T2D related complications, etc.

As such, in an embodiment of the present invention, there is provided a novel, genomic based classification tool for characterizing patients with higher risk for T2D complications. The use of such a classification tool can dramatically reduce the sample size (and/or the time and cost) required to perform clinical safety outcome studies in T2D. Such outcome studies are typically utilized in the clinical trial setting, and can also be utilized in animal testing.

As used herein, the term “clinical trial” means any research study designed to collect clinical data on responses to a particular treatment, and includes but is not limited to phase I, phase II and phase III clinical trials. Standard methods are used to define the patient population and to enroll subjects. Preferably, the clinical trials of the hereinbefore described embodiment of the instant invention relate to T2D and complications thereof, such as, for example, cardiovascular death, micro/macrovascular disorder, hypertension, neuropathy, atrial fibrillation, nephropathy or MACE, and the like.

Illustrative results include the following:

Identification of over 1000 SNPs highly significantly associated (p<10⁻³) with T2D complications.

Identification of Risk and Protective Alleles

Identification of genes and pathways associated with a specific or combined T2D complications

Determination that each most associated SNP alone has a low predictive value, while the combination of the SNPs provides a good predictive value for T2D complications.

In an embodiment of the present invention, there is provided a method for correlating a genetic feature with an increased or reduced risk of developing a complication associated with type-2 diabetes (T2D) and utilization of such information in the recruitment of subjects in clinical trials. Such complications include, but are not limited to, micro/macrovascular disorder, hypertension, neuropathy, atrial fibrillation, nephropathy or MACE or a combination thereof.

Preferably, under this embodiment, the complication is micro/micro/macrovascular complications, hypertension, atrial fibrillation, neuropathy or MACE.

The genetic feature is preferably a SNP or a STR, or a combination thereof.

In this embodiment, it is particularly preferable to detect a genetic feature which is

(a) at least one single nucleotide polymorphism (SNP) listed in Tables 1-4;
(b) at least one SNP which is in linkage disequlibrium with at least one SNP of (a); or
(c) at least one short tandem repeat (STR) that is in linkage disequilibrium with at least one SNP of (a).

According to an embodiment of the present invention, there is thus provided a method for characterizing a subject for inclusion or exclusion from a clinical trial, comprising detecting, in a sample obtained from said subject, the presence or absence of at least one genetic feature which is

(a) at least one single nucleotide polymorphism (SNP) listed in Tables 1-4;
(b) at least one SNP which is in linkage disequlibrium with at least one SNP of (a); or
(c) at least one short tandem repeat (STR) that is in linkage disequilibrium with at least one SNP of (a).

In particular embodiments of the invention, linkage disequilibrium (LD) is defined by a specific quantitative cutoff. As described in detail herein, linkage disequilibrium can be quantitatively determined by measures such as r2 and |D′|. As a consequence, certain embodiments of the invention relate to substitute markers in linkage disequilibrium by a measure within a certain range specified by particular values of r2 and/or |D′|. In an embodiment, LD is characterized by numerical values for r2 of greater than 0.1. In another embodiment, LD is characterized by numerical values for r2 of greater than 0.5. In another embodiment, LD is characterized by numerical values for r2 of greater than 0.8. In another embodiment, LD is characterized by numerical values for r2 of 0.9 or more. Other cutoff values for r2 are also contemplated, as described in more detail herein. In certain embodiments, LD is characterized by certain cutoff values for r2 and/or |D′|. In one such embodiment, LD is characterized by values for r2 and/or |D′| of greater than 0.2 and 0.8, respectively. Other combinations and permutations of these or other measures of LD are possible to practice the invention, and are also contemplated and within scope of the invention.

Preferably, the methods of the hereinbefore described embodiments of the instant invention involve detection of one or more of the aforementioned genetic features using techniques that are known in the art, such as those disclosed in the Examples. The present invention can also be practiced by using a wide variety of techniques and reagents which are known in the art for detecting the absence of the aforementioned genetic features, for example, using probe sequences that detect wild-type nucleic acid sequences.

In a more preferred embodiment, there is provided a method for selecting a patient for clinical trials comprising detecting in a biological sample of said patient, the presence or absence of at least one SNP listed in Tables 1-4, said SNP being selected on the basis of its p value of association with a complication, allele frequency, or odds ratio.

Under a related embodiment of the present invention, there is provided a method for characterizing patients for clinical trials based on the detection of a combination of biomarkers.

In such methods, better characterization of the patient cohort can be achieved. Any combinations of SNP listed in Tables 1-4 may be detected. Such combinations can be developed on the basis of, for example, level of association with a complication of interest and on the frequency of other genetic features. Such other genetic features, for example, risk-prone or protective alleles in the population, etc. may be included. Several methods are known by those skilled in the art to select appropriate markers.

In such embodiments of the instant invention wherein a combination of SNPs is detected, it is particularly preferable to employ a combination of biomarkers provided in each of Tables 1-4 (e.g., one SNP from each gene selected from the group consisting of PDE4D, SOX5, DMD, and SYT2).

In such embodiments, any two, any four, any five, any ten, any twenty, or more of the SNPs listed in Tables 1-4 may be detected.

The compositions and methods of the present invention also provide methods for reducing the cost and time for anti-diabetic drug development by “enriching” the outcome trial pool with pre-selected patients that are at greater risk of T2D-related complications. To this end, the present application describes methods for calculating a Risk Index Score, which combines clinical/biological biomarkers with genomic markers with high predictive performance. Such risk scores allow identification of a population subset with a higher complication rate. The Risk Index Score, can be optionally integrated into a Clinical Research Tool, thus facilitating evaluation of efficacy/safety balance in T2D by improving the signal to noise ratio.

In a related embodiment, the present application relates to kits and combinations that allow for practicing one or more of the aforementioned methods.

In this embodiment, there is provided combinations and kits for identifying a subject for clinical trial, wherein said subject is affected by type-2 diabetes (T2D) comprising in one or more packages
(a) an oligonucleotide that specifically hybridizes to a SNP having the RefSNP ID listed in Tables 1-4; or
(b) an oligonucleotide which is the complement of (a);
and one or more reagents for the detection of said oligonucleotide.

Methods of Therapy

The present invention discloses novel methods for the prevention and treatment of a T2D-related complication. In particular, the invention relates to methods of treatment of T2D-related complications. The term, “treatment” as used herein, refers not only to ameliorating symptoms associated with the disease, but also preventing or delaying the onset of the complication, and also lessening the severity or frequency of symptoms of the disease, preventing or delaying the occurrence of a second episode of the disease or condition; and/or also lessening the severity or frequency of symptoms of the disease or condition.

The present invention encompasses methods of treatment (prophylactic and/or therapeutic) for a T2D-related complication using a therapeutic agent. A “therapeutic agent” is an agent that alters (e.g., enhances or inhibits) enzymatic activity or function of a risk gene such as those disclosed in Tables 1-4 and/or expression of polymorphisms disclosed in Tables 1-4 and/or the specific metabolic or other biologically related pathway implicating those genes. The modes of useful therapeutic agents are further disclosed.

Representative therapeutic agents of the invention comprise the following: (a) nucleic acids, fragments, variants or derivatives of the genes, nucleic acids, or an active fragment or a derivative thereof and nucleic acids modifying the expression of said genes (e.g. antisense polynucleotides, catalytically active polynucleotides (e.g. ribozymes and DNAzymes), molecules inducing RNA interference (RNAi) and micro RNA), and vectors comprising said nucleic acids; (b) polypeptides, active fragments, variants or derivatives thereof, binding agents of polypeptides; peptidomimetics; fusion proteins or prodrugs thereof, antibodies; (c) metabolites of the gene products; (d) small molecules and compounds that alter (e.g., inhibit or antagonize) a risk gene expression, activity and/or function of a polypeptide encoded by said genes and; (e) small molecules and compounds that alter (e.g. induce, agonize or modulate) the expression or activity of said genes.

The nucleic acid sequences of T2D-related complication associated risk genes such as those disclosed in Tables 1-4 and/or polymorphisms disclosed in Tables 1-4 of this application are publicly available and can be used to design and develop therapeutic nucleic acid molecules and recombinant DNA molecules for the prevention and treatment of T2D or a T2D related condition. For example antisense nucleic acid molecules targeted to a polymorphism in Tables 1-4 can be designed using tools and the nucleotide sequence of the gene available in the art and constructed using chemical synthesis and/or enzymatic ligation reactions using procedures known in the art. Antisense nucleic acid molecule can be chemically synthesized using naturally occurring nucleotides or modified nucleotides designed to increase the biological stability of the molecules or to increase the physical stability of the duplex formed between the antisense oligonucleotide and sense nucleic acids, e.g., phosphorothioate derivatives and acridine substituted nucleotides can be used.

Alternatively, the antisense nucleic acid molecule can be produced biologically using an expression vector into which a nucleic acid molecule encoding a T2D-related complication risk gene, a fragment or a variant thereof has been cloned in antisense orientation (i.e., RNA transcribed from the expression vector will be complementary to the transcribed RNA of a T2D-related complication risk gene of interest).

More than one T2D-related complication therapeutic agent can be used concurrently, if desired. The therapy is designed to affect 1) expression of a T2D-related complication gene in a sense of activation, inhibition or modulation, 2) abundance, stability, biological activity and/or function of a T2D-related complication risk gene-encoded ribonucleic acid or polypeptide, or 3) biological activity and/or function of a T2D-related complication gene related signaling or metabolic pathway. Upregulation or increasing expression of a T2D-related complication risk gene or a particular variant of a T2D-related complication risk gene could interfere with or compensate for the expression or activity of a defective gene or variant; downregulation or decreasing expression or availability of a native risk gene or a particular splicing variant of a T2D-related complication susceptibility gene could minimize the expression or activity of a defective gene or the particular variant and thereby minimize the impact of the defective gene or the particular variant. The risk genes of the present invention are PDE4D, SOX5, DMD and SYT2. Variants thereof and isoforms thereof have been described hereinbefore.

The T2D and T2D-related complication therapeutic agent(s) are administered in a therapeutically effective amount that can be determined using established clinical methods and assays. The precise dose to be employed in the formulation will also depend on the route of administration, and the seriousness of the disease or disorder, and should be decided according to the judgment of a practitioner. Effective doses may be extrapolated from dose-response curves derived from in vitro or animal model test systems.

In one embodiment, a nucleic acid encoding a T2D-related complication polypeptide, fragment, variant or derivative thereof, either by itself or included within a vector, can be introduced into cells of an individual affected by T2D or a T2D related condition using variety of experimental methods described in the art, so that the treated cells start to produce native T2D-related complication susceptibility polypeptide. Thus, cells which, in nature, lack of a native T2D-related complication risk gene expression and activity, or have abnormal T2D-related complication risk gene expression and activity, can be engineered to express a T2D-related complication polypeptide or an active fragment or a different variant of said T2D-related complication susceptibility polypeptide. Genetic engineering of cells may be done either “ex vivo” (i.e. suitable cells are isolated and purified from a patient and re-infused back to the patient after genetic engineering) or “in vivo” (i.e. genetic engineering is done directly to a tissue of a patient using a vehicle). Alternatively, in another embodiment of the invention, a nucleic acid (e.g. a polynucleotide) which specifically hybridizes to the mRNA and/or genomic DNA of a T2D-related complication gene is administered in a pharmaceutical composition to the target cells or said nucleic acid is generated “in vivo”. The antisense nucleic acid that specifically hybridizes to the mRNA and/or DNA inhibits expression of the T2D-related complication polypeptide, e.g., by inhibiting translation and/or transcription. Binding of the antisense nucleic acid can be due to conventional base pairing, or, for example, in the case of binding to DNA duplexes, through specific interaction in the major groove of the double helix. In a preferred embodiment nucleic acid therapeutic agents of the invention are delivered into cells that express one or more T2D-related complication risk genes. A number of methods including, but not limited to, the methods known in the art can be used for delivering a nucleic acid to said cells. For example, a vector can be introduced in vivo such that it is taken up by a cell and directs the transcription of a RNA molecule, which induces RNA interference in the cell. Such a vector can remain episomal or become chromosomally integrated, and as long as it can be transcribed to produce the desired RNA molecules it will modify the expression of a T2D-related complication risk gene. Such vectors can be constructed by various recombinant DNA technology methods standard in the art.

The expression of an endogenous T2D-related complication risk gene can be reduced by inactivating completely (“knocking out”) or partially (“knocking down”) a T2D-related complication gene using targeted homologous recombination methods described in the art. Alternatively, expression of a functional, non-mutant T2D-related complication can be increased using a similar method: targeted homologous recombination can be used to replace a non-functional T2D-related complication risk gene with a functional form of the said gene in a cell. In another embodiment of the invention, other T2D therapeutic agents as described herein can also be used in the treatment or prevention of T2D or a related condition. The therapeutic agents can be delivered in a pharmaceutical composition; they can be administered systemically, or can be targeted to a particular tissue. The therapeutic agents can be produced by a variety of means, including chemical synthesis, cell culture and recombinant techniques (e.g. with transgenic cells and animals). Therapeutic agents can be isolated and purified to fulfill pharmaceutical requirements using standard methods described in the art. A combination of any of the above methods of treatment (e.g., administration of non-mutant T2D-related complication susceptibility polypeptide in conjunction with RNA molecules inducing RNA interference targeted to the mutant T2D-related complication susceptibility mRNA) can also be used.

In the case of pharmaceutical therapy the invention comprises compounds which affect 1) expression of a T2D-related complication gene in a sense of activation, inhibition or modulation, 2) abundance, stability, biological activity and/or function of a T2D-related complication risk gene-encoded ribonucleic acid or polypeptide, or 3) biological activity and/or function of a T2D-related complication gene or metabolic pathway encoded by T2D complication-associated risk genes such as those disclosed in Tables 1-4 and/or polymorphisms disclosed in Tables 1-4 of this application. The treatment may also exert its effects as specified above on one or several genes selected from the T2D complication-associated risk genes such as those disclosed in Tables 1-4 and/or polymorphisms disclosed in Tables 1-4 of this application.

Furthermore, a disclosed method or a test based on biomarkers specific for T2D-related complication susceptibility gene is useful in selection, modification or optimalization of therapeutic modalities for T2D-patients. For example if the less frequent, i.e. the minor, assumable mutated allele in the T2D-related complication susceptibility gene is risk-reducing, and if said mutation is a gene function reducing mutation, one can deduce that the gene function and/or activity would increase the risk of T2D complication. On that basis, drugs and other therapies such as gene therapies that reduce or inhibit the function or activity of the T2D-related complication susceptibility gene or the encoded protein would reduce the risk of the said T2D-related complication and could be used to both prevent and treat the said T2D-related complication in subjects having said mutated allele.

In another embodiment of the invention a T2D or T2D-related complication therapeutic agent comprises a known therapeutic agent related to a T2D-related complication associated gene listed in Tables 1-4 of this invention but which is not used to treat T2D or a T2D-related complication. Such compounds and therapeutic agents are useful for developing new therapies for T2D or a T2D-related complication as they most likely affect 1) expression of a T2D-related complication gene in a sense of activation, inhibition or modulation, 2) abundance, stability, biological activity and/or function of a T2D-related complication risk gene-encoded ribonucleic acid or polypeptide, or 3) biological activity and/or function of a T2D-related complication gene related signaling or metabolic pathway. These agents may be used alone or in combination with other treatments and agents used for prevention or treatment of T2D or a T2D-related condition.

In one embodiment of the invention, therapeutic agents or compounds currently utilized for the treatment of T2D and T2D-related complications are combined with one or more known therapeutic agents used to treat T2D comprising I. oral antidiabetics including biguanid derivatives such as 1) metformin, 2) buformin, insulin secretagogues such as 1) sulphonylurea derivatives such as tolbutamide, glibenclamide, gliclazide, glipizide, glimepiride, gliquidone; 2) meglitinides such as repaglinide, nateglinide; 3) alpha-glucosidase inhibitors such as acarbose, miglitol; 4) thiazolidinediones such as rosiglitazone and pioglitazone; 5) other defined by World Health Organization—The Anatomical Therapeutic Chemical (ATC) classification system; II. insulin such as i) insulin glargine, ii) insulin aspart, iii) insulin lispro, iv) insulin glulisine; v) insulin detemir; and agents known do decrease and or prevent diabetes related complication, such as high blood pressure, i) converting enzyme inhibitors, ii) angiotensin receptor blockers, iii) direct renin inhibitors, iv) endothelin antagonists, v) diuretics, vi) beta blockers, vii) alpha blockers, viii) inhibitors of phosphodiesterase 5a and the combinations thereof.

Pharmaceutical Compositions

The present invention also pertains to pharmaceutical compositions comprising agents described herein, particularly polynucleotides, polypeptides and any fractions, variants or derivatives of T2D-related complication genes, and/or agents that alter (e.g., enhance or inhibit) expression of a risk gene or genes, or activity of one or more polypeptides encoded by associated genes as described herein. For instance, an agent that alters expression of a risk gene or activity of one or more polypeptides encoded thereby.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents. The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration. For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of a T2D-related complication, and should be decided according to the judgment of a practitioner and each patient's circumstances. Effective doses may be extrapolated from dose-response curves derived from in vitro or animal model test systems.

Functional Foods

By definition “functional foods” or “nutraceuticals” are foods or dietary components or food ingredients that may provide a health benefit beyond basic nutrition. Functional foods are regulated by authorities (e.g. by the FDA in US) according to their intended use and the nature of claims made on the package. Functional foods can be produced by various methods and processes known in the art including, but not limited to synthesis (chemical or microbial), extraction from a biological material, mixing functional ingredient or component to a regular food product, fermentation or using a biotechnological process. A functional food may exert its effects directly in the human body or it may function e.g. through human intestinal bacterial flora.

The associated genes disclosed in Tables 1-4 of this invention can be used as molecular targets towards which functional foods claiming health benefit in a T2D related complication can be developed. For example a functional food may compensate reduced biological activity of a polypeptide encoded by a gene set forth in Tables 1-4 when the risk gene is defective or is not expressed properly in a subject. A functional food may also inhibit the expression and/or biological activity of a gene or polypeptide of the invention promoting the development of a T2D related complication. In another embodiment a functional food may increase the expression and/or biological activity of a gene or polypeptide protecting an individual from the development of a T2D related complication due to reduced expression and protein production.

Aspects of the instant invention include, but are not limited to:

Aspect 1. A method for predicting a risk of developing a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject affected with type-2 diabetes (T2D), comprising detecting, in a sample obtained from said subject, at least one genetic feature which is single nucleotide polymorphism (SNP) or short tandem repeat (STR),

wherein the detection of said genetic feature in said subject correlates with said risk of developing at least one of said complication.

Aspect 2. The method according to aspect 1, wherein said SNP comprises a polymorphism of a gene or a locus linked thereto.
Aspect 3. The method according to aspect 1, wherein said genetic feature comprises at least one feature listed in Table 1-4.
Aspect 4. The method according to aspect 1, wherein said genetic feature is at least one SNP having the RefSNPID (rs) rs11951359, rs12657171, rs294492, rs294494, rs16889508, rs7880606, rs808517, rs808521, rs1379106, rs5972470, rs2692986, rs5972687, rs16927597, rs16926892, rs7963345, rs2728841, rs6698441, rs4537626, rs7517181 or rs12404969.
Aspect 5. The method according to aspect 1, wherein said genetic feature is at least one SNP or one STR found to be in linkage disequilibrium with a SNP having the RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441
Aspect 6. The method according to aspect 1, comprising detecting a STR of at least one gene which is Phosphodiesterase 4D, cAMP-specific (PDE4D; NCBI Gene ID: 5144), Dystrophin (DMD; NCBI Gene ID: 1756), sex determining region Y (SRY)-box5 (SOX5; NCBI Gene ID: 6660), Synaptotagmin II (SYT2; or NCBI Gene ID: 127833).
Aspect 7. The method according to aspect 1, wherein detection of said genetic feature correlates with increased or reduced risk of developing said complication.
Aspect 8. A method for predicting a risk of developing a complication which is micro/macrovascular disorder, hypertension, neuropathy, atrial fibrillation, nephropathy or other major adverse cardiovascular events (MACE) in a subject affected by type II diabetes, comprising

detecting,
in a sample obtained from said subject, at least one single nucleotide polymorphism (SNP) having the RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441 ;
   or
   one STR found to be in linkage disequilibrium with a gene which is Phosphodiesterase 4D, cAMP-specific (PDE4D; NCBI Gene ID: 5144), Dystrophin (DMD; NCBI Gene ID: 1756), sex determining region Y (SRY)-box5 (SOX5; NCBI Gene ID: 6660), Synaptotagmin II (SYT2; or NCBI Gene ID: 127833),

wherein the detection of said SNP or STR in said subject correlates with said risk of developing said complication.

Aspect 9. A method for predicting a risk of developing a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject affected by T2D, comprising detecting, in a sample obtained from said subject, at least one (SNP) from at least on gene which is Phosphodiesterase 4D, cAMP-specific (PDE4D; NCBI Gene ID: 5144), Dystrophin (DMD; NCBI Gene ID: 1756), sex determining region Y (SRY)-box5 (SOX5; NCBI Gene ID: 6660), Synaptotagmin II (SYT2; NCBI Gene ID: 127833) or at least one STR found to be in linkage disequilibrium with at least one (SNP) of said gene, wherein the detection of said SNP or STR in said subject correlates with said risk of developing said complication.
Aspect 10: A method for predicting the risk of developing a complication which is micro/macrovascular disorder, hypertension, neuropathy, atrial fibrillation, nephropathy or other major adverse cardiovascular events (MACE), comprising detecting at least one SNP having RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441, said SNP being selected on the basis of its p value of association with said complication(s), allele frequency and odds ratio.
Aspect 11. The method according to aspects 8 or 9 or 10, comprising detecting at least two SNPs.
Aspect 12. The method according to aspects 8 or 9 or 10, comprising detecting at least three SNPs.
Aspect 13. The method according to aspects 8 or 9 or 10, comprising detecting more than three SNPs
Aspect 14. The method according to aspects 8 or 9 or 10, wherein comprising detecting at least one SNP associated with Phosphodiesterase 4D, cAMP-specific (PDE4D; or NCBI Gene ID: 5144).
Aspect 15: The method of aspect 14, wherein the SNP is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190
Aspect 16. The method according to aspect 1, wherein the genetic feature is
(a) at least one single nucleotide polymorphism (SNP) having RefSNPID rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866 or rs2095981, rs4950867, rs4453099 or rs6698441;
(b) at least one SNP which is in linkage disequilibrium with at least one SNP of (a); or
(c) short tandem repeat (STR) that is in linkage disequilibrium with at least one SNP of (a).
   Aspect 17. The method according to Aspect 16, comprising detecting at least two SNPs.
   Aspect 18. The method according to Aspect 16, comprising detecting at least three SNPs.
   Aspect 19. The method according to aspect 1, with the proviso that said at least one SNP which is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492 or rs893190 is detected.
   Aspect 20. The method according to aspect 2, with the proviso that said at least two SNPs from the group consisting of rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492 or rs893190 are detected.
   Aspect 21: The method according to aspect 1, wherein the complication is hypertension and the method comprises detecting at least one SNP which is rs11951359 (PDE4D), rs1379106 (DMD), rs5972470 (DMD), rs2692986 (DMD), rs2728841 (SOX5) rs4537626 (SYT2), rs7517181 (SYT2) or rs12404969 (SYT2).
   Aspect 22: The method according to aspect 1, wherein the complication is micro-macro vascular disorders and the method comprises detecting at least one SNP which is rs11951359 (PDE4D), rs12657171 (PDE4D) rs5972687 (DMD), rs2692986 (DMD), rs2728841 (SOX5) rs4537626 (SYT2), rs7517181 (SYT2) or rs12404969 (SYT2).
   Aspect 23: The method according to aspect 1, wherein the complication is MACE and the method comprises detecting at least one SNP which is rs11951359 (PDE4D).
   Aspect 24: The method according to aspect 1, wherein the complication is a combination of MACE and albuminuria and the method comprises detecting at least one SNP which is rs11951359 (PDE4D) and rs12657171 (PDE4D).
   Aspect 25: The method according to aspect 1, wherein the complication is atrial fibrillation and the method comprises detecting at least one SNP which is rs294492 (PDE4D), rs294494 (PDE4D), rs7880606 (DMD), rs808517 (DMD), rs808521 (DMD), rs16927597 (SOX5), rs16926892 (SOX5) or rs7963345 (SOX5).
   Aspect 26: The method according to aspect 1, wherein the complication is neuropathy and the method comprises detecting at least one SNP which is rs16889508 (PDE4D).
   Aspect 27. The method according to any one of aspects 21, 22, 24 and 25, comprising detecting two or more SNPs.
   Aspect 28. A kit for predicting a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject affected by type-2 diabetes (T2D) comprising in one or more packages
(a) an oligonucleotide that specifically hybridizes to a SNP RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441; or
(b) an oligonucleotide which is the complement of (a);
and one or more reagents for the detection of said oligonucleotide.
   Aspect 29. The kit according to aspect 21, comprising one or more reagents for polymerase chain reaction (PCR).
   Aspect 30. The kit of aspect 28, further comprising an RNase.
   Aspect 31. The kit of aspect 28, which further comprises one or more reagents for isolation of cells from a sample.
   Aspect 32. The kit of aspect 31, wherein said sample is a blood sample.
   Aspect 33. The kit of aspect 28, further comprising a DNAse inhibitor.
   Aspect 34. The kit of aspect 28, further comprising reagents for sequencing in one or more packages.
   Aspect 35. The kit of aspect 28, further comprising a cDNA microarray plate in one or more packages.
   Aspect 36. The kit of aspect 28, wherein the oligonucleotide is a DNA which hybridizes to said SNP having a sequence which is disclosed in the NCBI single nucleotide polymorphism database.
   Aspect 37. The kit of aspect 28, wherein the oligonucleotide is
   (a) an oligonucleotide comprising at least 80% sequence identity to the oligonucleotide having the RefSNP ID which is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441
   (b); or
   (c) an oligonucleotide which is complimentary to the oligonucleotide of (a).
      Aspect 38. The kit of aspect 37, wherein the oligonucleotide is
   (d) an oligonucleotide comprising at least 90% sequence identity to the oligonucleotide having the RefSNP ID which is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441; or
   (e) an oligonucleotide which is complimentary to the oligonucleotide of (a).
      Aspect 39. The kit of aspect 37, wherein the oligonucleotide is
   (f) an oligonucleotide comprising at least 95% sequence identity to the oligonucleotide having the RefSNP ID which is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441; or
   (g) an oligonucleotide which is complimentary to the oligonucleotide of (a).
      Aspect 40. The kit of aspect 37, wherein the oligonucleotide is
   (h) an oligonucleotide comprising at least 100% sequence identity to the oligonucleotide having the RefSNP ID which is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441
   (i); or
   (j) an oligonucleotide which is complimentary to the oligonucleotide of (a).
      Aspect 41. The kit of aspect 28, which further comprises a control oligonucleotide that specifically hybridizes to the wild-type allele.
      Aspect 42. Use of a kit according to Aspect 28 for predicting a complication which is micro/macrovascular disorder, hypertension, neuropathy, atrial fibrillation, nephropathy or a major adverse cardiovascular events (MACE) in a subject affected by type-2 diabetes (T2D).
      Aspect 43. A method for preventing, treating or reducing the risk of T2D or a T2D related complication which is micro/macrovascular disorder, hypertension, neuropathy, atrial fibrillation, nephropathy or a major adverse cardiovascular events (MACE) in a subject in need thereof, comprising administrating to said subject a therapeutic agent which
(a) alters the enzymatic activity or levels of a polypeptide encoded by at least one risk gene listed in Tables 1-4; or
(b) alters the expression of at least one polymorphism disclosed in Tables 1-4; or
(c) alters the specific metabolic or other biologically related pathway implicating the risk gene of (a).
   Aspect 44. The method according to aspect 43, wherein the therapeutic agent alters the expression of at least one polymorphism disclosed in Tables 1-4.
   Aspect 45. The method according to aspect 44, wherein the therapeutic agent is an antisense oligonucleotide or an siRNA.
   Aspect 46. The method according to aspect 43, wherein the therapeutic agent alters the levels or enzymatic activity of a polypeptide encoded by at least one risk gene listed in Tables 1-4.
   Aspect 47. The method according to aspect 46, wherein the therapeutic agent is an antibody.
   Aspect 48. A method for identifying a subject for preventive therapeutic action, comprising detecting, in a sample obtained from said subject, at least one genetic feature which is
   (a) at least one single nucleotide polymorphism (SNP) listed in Tables 1-4;
   (b) at least one SNP which is in linkage disequilibrium with at least one SNP of (a); or
   (c) short tandem repeat (STR) that is in linkage disequilibrium with at least one SNP of (a),
   wherein the detection of said genetic feature in said subject correlates with the eligibility of said subject for said preventive therapeutic action.

An embodiment of the present invention also relates to the following aspects:

Aspect A. A method for predicting a risk of developing a complication which is micro/macrovascular disorder, hypertension, neuropathy, atrial fibrillation, nephropathy, albuminuria or a major adverse cardiovascular event (MACE) in a subject affected with type-2 diabetes (T2D), comprising detecting, in a sample obtained from said subject, at least one genetic feature which is single nucleotide polymorphism (SNP) of the gene phosphodiesterase (PDE4D), wherein

detection of one or more SNPs having the RefSNPID rs12657171 and rs11951359 is associated with increased likelihood of developing micro and micro/macrovascular disorders, MACE or albuminuria;

detection of a SNP having the RefSNPID rs11951359 or rs11951359 is associated with increased likelihood of developing MACE or hypertension;

detection of one or more SNPs having the RefSNPID rs294492 and rs294494 is associated with increased likelihood of developing atrial fibrillation; and

detection of a SNP having the RefSNPID rs16889508 is associated increased likelihood of developing neuropathy.

Aspect B. A method for predicting a risk of developing a complication which is micro/macrovascular disorder, hypertension or atrial fibrillation in a subject affected with type-2 diabetes (T2D), comprising detecting, in a sample obtained from said subject, at least one genetic feature which is single nucleotide polymorphism (SNP) of the gene dystrophin (DMD), wherein

detection of a SNP having the RefSNPID rs16889508 is associated with increased likelihood of developing atrial fibrillation or hypertension;

detection of one or more SNPs having the RefSNPID rs808517 and rs808521 is associated with increased likelihood of developing atrial fibrillation;

detection of one or more SNPs having the RefSNPID rs5972470 and rs2692986 is associated with increased likelihood of developing hypertension; and

detection of one or more SNPs having the RefSNPID rs5972687 and rs2692986 is associated with increased likelihood of developing micro-macrovascular disorders.

Aspect C. A method for predicting a risk of developing a complication which is micro/macrovascular disorder, hypertension or atrial fibrillation in a subject affected with type-2 diabetes (T2D), comprising detecting, in a sample obtained from said subject, at least one genetic feature which is single nucleotide polymorphism (SNP) of the gene SOX5, wherein

detection of a SNP having the RefSNPID rs2728841 is associated with increased likelihood of developing both macro/micro vascular disorders and hypertension; and

detection of one or more SNPs having the RefSNPID rs16927597, rs16926892 and rs7963345 is associated with increased likelihood of developing atrial fibrillation.

Aspect D. A method for predicting a risk of developing a complication which is micro/macrovascular disorder, hypertension or atrial fibrillation in a subject affected with type-2 diabetes (T2D), comprising detecting, in a sample obtained from said subject, at least one genetic feature which is single nucleotide polymorphism (SNP) of the gene SYT2, wherein

detection of a SNP having the RefSNPID rs4537626 is associated with increased likelihood of developing macro/micro vascular disorders and hypertension;

detection of one or more SNPs having the RefSNPID rs7517181 and rs12404969 is associated with increased likelihood of developing hypertension;

detection of one or more SNPs having the RefSNPID rs7517181 and rs12404969 is associated with increased likelihood of developing micro-macro vascular complications; and

detection of a SNP having the RefSNPID rs6698441 is associated with increased likelihood of developing atrial fibrillation.

Aspect E. A method for predicting a risk of developing a complication which is hypertension in a subject affected with type-2 diabetes (T2D), comprising detecting, in a sample obtained from said subject, at least one genetic feature which is single nucleotide polymorphism (SNP) having the RefSNPID rs11951359 of the gene PDE4D, rs1379106 of the gene DMD, rs5972470 of the gene DMD, rs2692986 of the gene DMD, rs2728841 of the gene SOX5, rs4537626 of the gene SYT2, rs7517181 of the gene SYT2 or rs12404969 of the gene SYT2, wherein detection of said SNP is associated with increased likelihood of developing said hypertension.
Aspect F. A method for predicting a risk of developing a complication which is micro-macro vascular disorders in a subject affected with type-2 diabetes (T2D), comprising detecting, in a sample obtained from said subject, at least one genetic feature which is single nucleotide polymorphism (SNP) having the RefSNPID rs11951359 of the gene PDE4D, rs12657171 of the gene PDE4D, rs5972687 of the gene DMD, rs2692986 of the gene DMD, rs2728841 of the gene SOX5, rs4537626 of the gene SYT2, rs7517181 of the gene SYT2 or rs12404969 of the gene SYT2, wherein detection of said SNP is associated with increased likelihood of developing said hypertension.
Aspect G. A method for predicting a risk of developing a complication which is MACE in a subject affected with type-2 diabetes (T2D), comprising detecting, in a sample obtained from said subject, at least one genetic feature which is single nucleotide polymorphism (SNP) having the RefSNPID rs11951359 of the gene PDE4D, wherein detection of said SNP is associated with increased likelihood of developing said MACE.
Aspect H. A method for predicting a risk of developing a complication which is a combination of MACE and albuminuria in a subject affected with type-2 diabetes (T2D), comprising detecting, in a sample obtained from said subject, at least one genetic feature which is single nucleotide polymorphism (SNP) having the RefSNPID rs11951359 of the gene PDE4D or rs12657171 of the gene PDE4D, wherein detection of said SNP is associated with increased likelihood of developing said combination of MACE and albuminuria.
Aspect I. A method for predicting a risk of developing a complication which is atrial fibrillation in a subject affected with type-2 diabetes (T2D), comprising detecting, in a sample obtained from said subject, at least one genetic feature which is single nucleotide polymorphism (SNP) having the RefSNPID rs294492 of the gene PDE4D, rs294494 of the gene PDE4D, rs7880606 of the gene DMD, rs808517 of the gene DMD, rs808521 of the gene DMD, rs16927597 of the gene SOX5, rs16926892 of the gene SOX5 or rs7963345 of the gene SOX5, wherein detection of said SNP is associated with increased likelihood of developing said atrial fibrillation.
Aspect J. A method for predicting a risk of developing a complication which is neuropathy in a subject affected with type-2 diabetes (T2D), comprising detecting, in a sample obtained from said subject, at least one genetic feature which is single nucleotide polymorphism (SNP) having the RefSNPID rs16889508 of the gene PDE4D, wherein detection of said SNP is associated with increased likelihood of developing said neuropathy.
Aspect K. A method according to aspects A-K, comprising detecting at least two SNPs.
Aspect L. A method according to aspects A-K, comprising detecting at least two different SNPs of two different genes selected from PDE4D, SOX5, DMD and SYT2.
Aspect M. A method according to aspects A-K, comprising detecting at least two different SNPs from a single gene selected from PDE4D, SOX5, DMD and SYT2.
Aspect N. A kit for predicting a complication which is micro/macrovascular disorder, hypertension, neuropathy, atrial fibrillation, nephropathy or a major adverse cardiovascular event (MACE) in a subject affected by type-2 diabetes (T2D) comprising in one or more packages
(a) two or more oligonucleotides that specifically hybridizes to two SNPs having RefSNPID (rs) rs11951359, rs12657171, rs294492, rs294494, rs16889508, rs7880606, rs808517, rs808521, rs1379106, rs5972470, rs2692986, rs5972687, rs16927597, rs16926892, rs7963345, rs2728841, rs6698441, rs4537626, rs7517181 or rs12404969; or
(b) two or more oligonucleotides that are complementary to the SNPs of (a); and one or more reagents for the detection of said oligonucleotide.
   Aspect O. The kit according to aspect N, wherein the oligonucleotides of (a) or (b) are arrayed in a microarray format.
   Aspect P. The kit according to aspect N, for predicting a risk of developing hypertension in a subject affected with type-2 diabetes (T2D) comprising in one or more packages
(a) an oligonucleotide that specifically hybridizes to one or more SNPs having the RefSNPID (rs) rs11951359 of the gene PDE4D, rs1379106 of the gene DMD, rs5972470 of the gene DMD, rs2692986 of the gene DMD, rs2728841 of the gene SOX5, rs4537626 of the gene SYT2, rs7517181 of the gene SYT2 or rs12404969 of the gene SYT2; or
(b) an oligonucleotide which is the complement of (a).
   Aspect Q. The kit according to aspect N, for predicting a risk of developing micro-macro vascular disorders in a subject affected with type-2 diabetes (T2D), comprising in one or more packages
(a) an oligonucleotide that specifically hybridizes to one or more SNPs having the RefSNPID (rs) rs11951359 of the gene PDE4D, rs12657171 of the gene PDE4D, rs5972687 of the gene DMD, rs2692986 of the gene DMD, rs2728841 of the gene SOX5, rs4537626 of the gene SYT2, rs7517181 of the gene SYT2 or rs12404969 of the gene SYT2; or
(b) an oligonucleotide which is the complement of (a).
   Aspect R. The kit according to aspect N, for predicting a risk of developing MACE in a subject affected with type-2 diabetes (T2D), comprising in one or more packages
(a) an oligonucleotide that specifically hybridizes to a SNP having the RefSNPID (rs) rs11951359 of the gene PDE4D; or
(b) an oligonucleotide which is the complement of (a).
   Aspect S. The kit according to aspect N, for predicting a risk of developing a combination of MACE and albuminuria in a subject affected with type-2 diabetes (T2D), comprising in one or more packages
(a) an oligonucleotide that specifically hybridizes to one or more SNPs having the RefSNPID (rs) rs11951359 of the gene PDE4D or rs12657171 of the gene PDE4D; or
(b) an oligonucleotide which is the complement of (a).
   Aspect T. The kit according to aspect N, for predicting a risk of developing atrial fibrillation in a subject affected with type-2 diabetes (T2D), comprising in one or more packages
(a) an oligonucleotide that specifically hybridizes to one or more SNPs having the RefSNPID (rs) rs294492 of the gene PDE4D, rs294494 of the gene PDE4D, rs7880606 of the gene DMD, rs808517 of the gene DMD, rs808521 of the gene DMD, rs16927597 of the gene SOX5, rs16926892 of the gene SOX5 or rs7963345 of the gene SOX5; or
(b) an oligonucleotide which is the complement of (a).
   Aspect U. The kit according to aspect N, for predicting a risk of developing neuropathy in a subject affected with type-2 diabetes (T2D), comprising in one or more packages
(a) an oligonucleotide that specifically hybridizes to one or more SNPs having the RefSNPID (rs) rs16889508 of the gene PDE4D; or
(b) an oligonucleotide which is the complement of (a).

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1. Genes associated to different outcomes. Numbers correspond to genes associated to one or more phenotypes at p-value<10⁻³.

FIG. 2. (A) Schematic structure of PDE4D gene on chromosome 5 showing the position of 29 SNPs associated to different phenotypes and (B) haploblocks from 58 264 kb to 59 784 kb (Genome Build 37.2 (hg19)) in CEU based on Haploview 4.2, V3, Release 27 demonstrating that different SNPs are located in different CEU haploblocks and associated to different phenotypes.

FIG. 3. (A) Schematic structure of SYT2 gene on chromosome 1 showing the position of 13 SNPs associated to different phenotypes and (B) haploblocks from 202 559 kb to 202 680 kb (Genome Build 37.2 (hg19)) in CEU based on Haploview 4.2, V3, Release 27 demonstrating that different SNPs are located in different CEU haploblocks and associated to different phenotypes.

FIG. 4. Schematic structure of SOX5 gene on chromosome 12 showing the position of 7 SNPs associated to different phenotypes. SOX gene is located from 23 685 kb to 24 716 kb (Genome Build 37.2 (hg19)).

FIG. 5. Schematic structure of DMD gene on chromosome X showing the position of 16 SNPs associated to different phenotypes. DMD gene is located from 31 137 kb to 33 358 kb (Genome Build 37.2 (hg19))

### BRIEF DESCRIPTION OF THE TABLES

Tables 1-4: Lists of common genes associated to 6 or more complications of T2D. Analyses were performed using the minor allele for each SNP, the minor allele being defined as the allele that was found the least frequent in the population under study.

Table 1 lists SNPs of PDE4D associated to 9 phenotypes with odds ratio and their positions.

Table 2 lists SNPs of DMD associated to 10 phenotypes with odds ratio and their positions.

Table 3 lists SNPs of SOX5 associated to 6 phenotypes with odds ratio and their positions.

Table 4 lists SNPs of SYT2 associated to 7 phenotypes with odds ratio and their positions.

Table 5 lists genes that are associated to 6 and more different phenotypes.

Without further elaboration, it is believed that one skilled in the art can, using the preceding description, utilize the following invention to its fullest extent. The following specific preferred embodiments are, therefore, to be construed as merely illustrative, and not limitative of the remainder of the disclosure in any way whatsoever.

In the forgoing and in the following examples, all temperatures are set forth uncorrected in degrees Celsius and, all parts and percentages are by volume, unless otherwise indicated.

### EXAMPLES

The invention will be explained below with reference to the following non-limiting examples.

### Example 1

### Methods

Total genomic DNA was extracted from human blood with FlexiGene DNA kit from Qiagen and dissolved in sterile TE buffer. DNA collection was preserved at a standard concentration of 1 μg/μl in a cold room. ADVANCE patients (n=2313) of Caucasian origin (Pritchard et al. Inference of population structure using multilocus genotype data. Genetics, 55:945-59, 2000) having several complications of T2D were compared to controls T2D patients without such complications, recruited for older age or long T2D duration. All patients' DNA was genotyped by Affymetrix GeneChip® SNP arrays 5.0 and 6.0. This assay is comprised of 1 array and two assay kits. The array is designed to detect over 906 000 single nucleotide polymorphism (SNPs) on the human genome plus 946 000 copy number variants. Genome-wide human SNP Nsp/Sty assay kit was used (Purcell et al. A toolset for whole-genome association and population-based linkage analysis. American Journal of Human Genetics, 81, 2007). For SNP mapping, genomic DNA was diluted with reduced EDTA TE buffer at a concentration of 50 ng/μl. The assay required 500 ng of genomic DNA. The principle of this assay is based on a strategy that reduces the complexity of the human genome by digesting DNA with Nspl and Styl restriction enzymes (RE), ligating RE specific sequences at the end of DNA fragments and preferentially amplifying 250-2000 base pairs amplicons of RE fragments by a single PCR primer. 50 μg of the purified PCR products are then submitted to fragmentation with DNase Ito a size of 20-100 base pairs, end-labelled and injected into SNP arrays 5.0 or 6.0 to be hybridised for 16 hours at 50° C. The arrays are washed and stained in the Affymetrix F-450 fluidics station in a three stage automated process consisting of a streptavidin-phycoerythin (SAPE) stain followed by a biotinylated anti-streptavidin antibody amplification and final stain with streptavidin-phycoerythin. Following staining, the arrays are filled with buffer and scanned with the Affymetrix GeneChip Scanner 3000 7G.

The automation of the fluidics station and the scanner is supported by the Affymetrix genotyping command console (AGCC) as well as the samples and experiment registration, the image acquisition and the image files exportation to genotyping console for further analysis. Genotyping console is a software application that allows the analysis and the evaluation of the data from the image of the array and generates genotype calls. It creates reports of the analysis and the data and allows the exportation of those reports and data to other software applications for biostatistical analysis.

The genome wide association study (GWAS) was done with PLINK v1.07 to generate the lists of SNPs associated to micro and micro/macrovascular complications, hypertension, major adverse cardiovascular events (MACE) or MACE+albuminuria, neuropathy and atrial fibrillation. Analyses were performed using the minor allele for each SNP, the minor allele being defined as the allele that was found the least frequent in the population under study. With some exception, the threshold of p-value of SNPs associated to our different outcomes was 10⁻³. The extraction of genes associated to phenotypes described above was done with NetAffx™ analysis center (available on the world-wide-web at affymetrix.com/analysis/index.affx). Using the list of SNPs generated by GWAS, we created several sub-lists of SNPs corresponding to each phenotype. These sub-lists of associated SNPs were submitted to the NetAffx™ Analysis Center to generate lists of genes. We retrieved and export the results from NetAffx™ to Excel sheet for comparison. We focused our analysis on SNPs within annotated genes.

Results

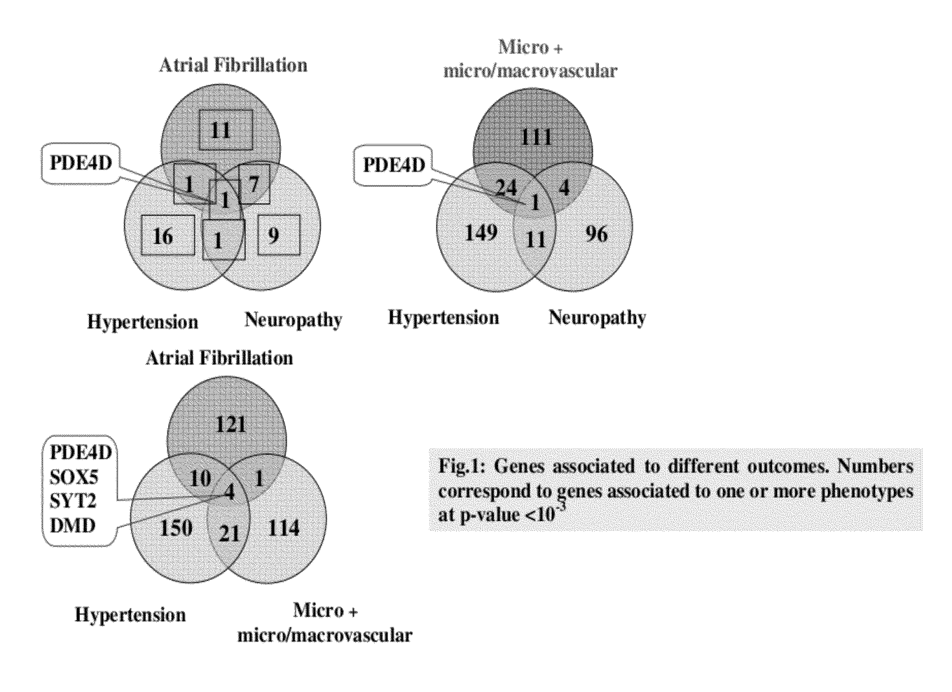

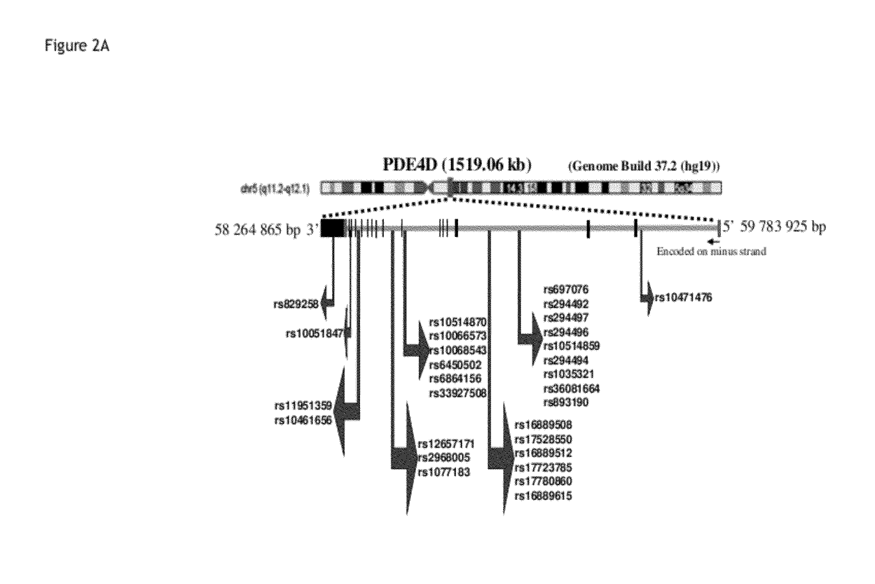

We observed 140, 185, 112, and 136 genes associated with micro-+macrovascular disorder, hypertension, neuropathy, and atrial fibrillation respectively in diabetic patients from ADVANCE cohort (FIG. 1). Table 5 lists the phenotypes that are associated to each of the four genes. PDE4D is the only gene found to be associated to a combination of 9 phenotypes: have_albuminuria, macrovascular disease, micro_+macrovascular disease, MI+angina, MACE, MACE+albuminuria, hypertension, atrial fibrillation and neuropathy (Table 5), while SYT2 and DMD are associated to 7 phenotypes and SOX5 is associated to 6 phenotypes. Five phenotypes (micro_+macrovascular disease, MACE, MACE+albuminuria, hypertension and atrial fibrillation) are common to the four genes.

TABLE 5
Genes associated to 6 and more different phenotypes.

| | PDE4D | SYT2 | SOX5 | DMD |
|---|---|---|---|---|
| Have_Albuminuria | X | | | |
| Microvascular | | | | X |
| Macrovascular | X | X | | |
| Micro_macrovascular | X | X | X | X |
| MI_Angina | X | X | X | |
| MACE | X | X | X | X |
| MACE_Albuminuria | X | X | X | X |
| Hypertension | X | X | X | X |
| Atrial Fibrillation | X | X | X | X |
| Neuropathy | X | | | X |

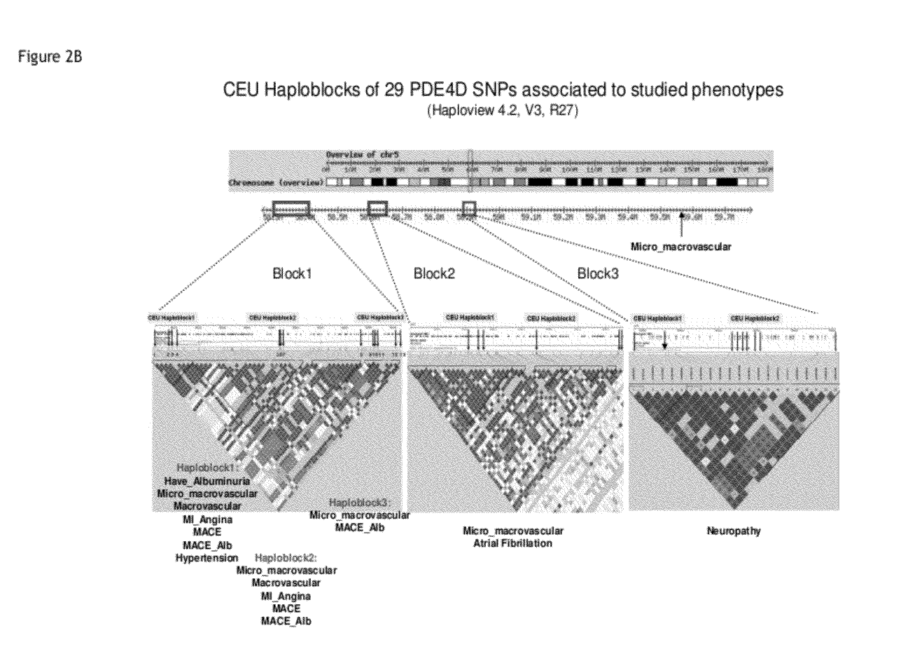

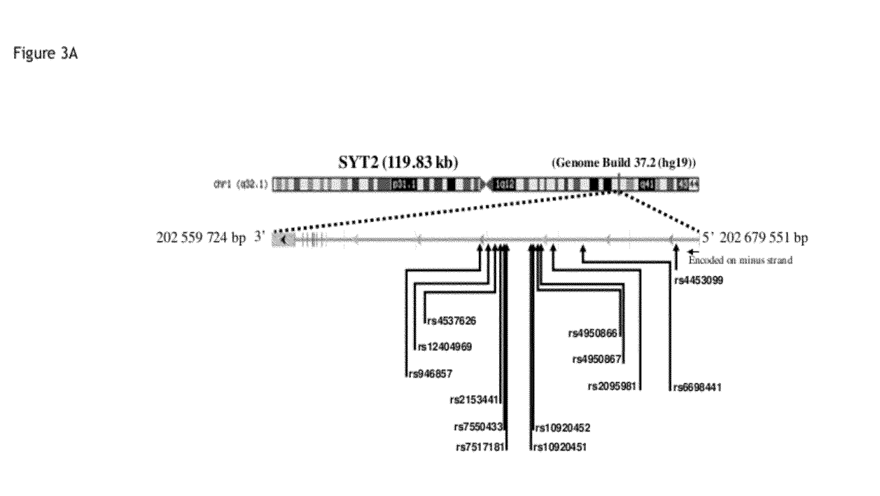

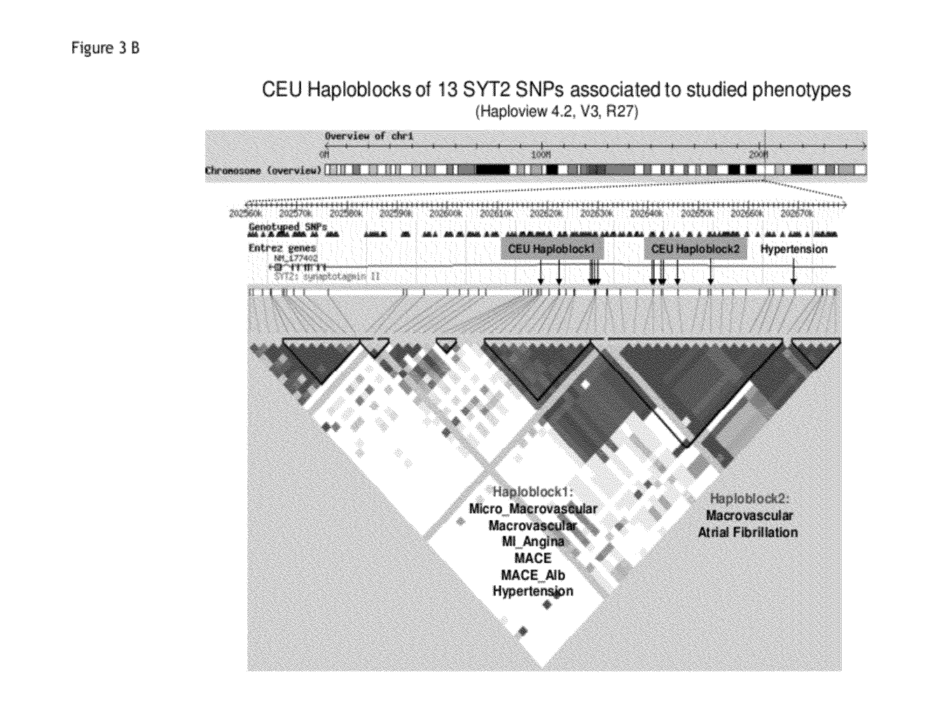

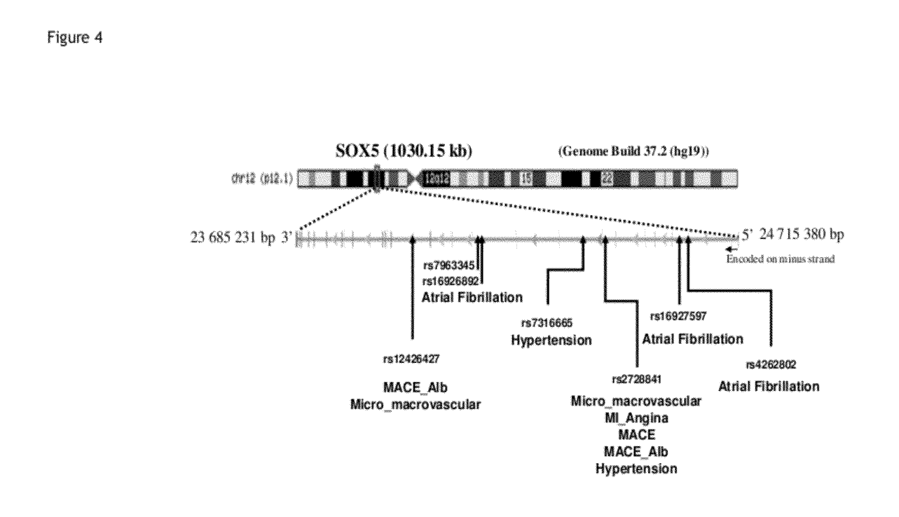

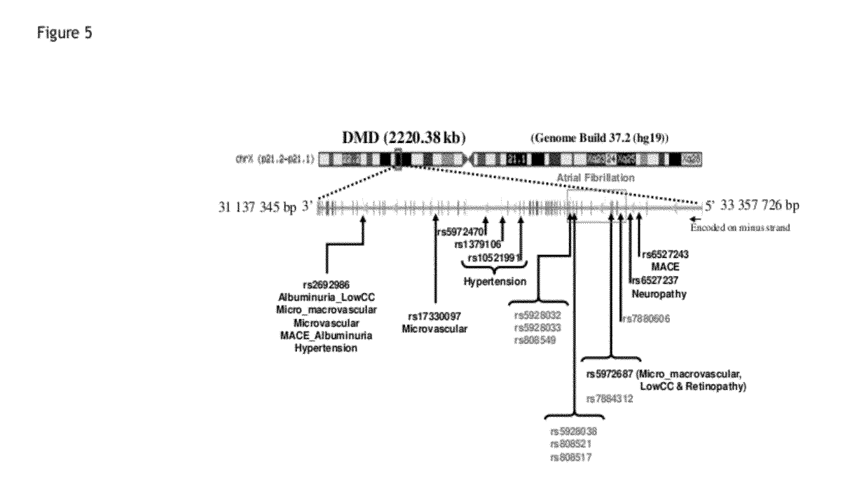

The comparison of these different gene lists show common and specific genes associated with different phenotypes PDE4D, phosphodiesterase 4D, cAMP-specific gene is located in human chromosome 5(q12) and encoded on minus strand (FIG. 1A). Genome Reference Consortium Human Build Primary_Assembly, GRCh37 from Pubmed allows us to define the positions and lengths of exons and introns constituting the PDE4D gene and also the positions of interest SNPs (FIG. 1A). From our previous lists of SNPs generated by PLINK v1.07, we can extract the SNPs ID of PDE4D gene associated with distinct complications. 28 SNPs are located within introns and one SNP is located in the 3′UTR of PDE4D gene (FIG. 1A). The first group of SNPs (rs829258, rs10051847, rs11951359, rs10461656, rs12657171, rs2968005, rs1077183, rs10514870, rs10066573, rs10068543, rs6450502, rs6864156 and rs33927508) residing within the same Block1, based on Haploview 4.2, V3, Release 27, were associated significantly (p<10⁻³) to have_albuminuria, micro_+macrovascular disease, macrovascular disease, MI+angina, MACE, MACE+albuminuria and hypertension. This block could be divided further into 3 sub-haploblocks (FIG. 1B). A second group of SNPs (rs16889508, rs17528550, rs16889512, rs17723785, rs17780860 and rs16889615) was associated to micro_+macrovascular disease and atrial fibrillation and located in the block2 while the last block (3) contains SNPs (rs697076, rs294492, rs294497, rs294496, rs10514859, rs294494, rs1035321, rs36081664 and rs893190) that are associated to neuropathy (FIG. 1B). Finally, the SNP rs10471476 is associated to micro_+macrovascular disease and is located at the 5′ end of the gene. These observations support the association results. In the literature, PDE4D gene was described essentially in stroke and our results show for the first time the involvement of this gene in other diabetes complications. In addition, 3 other genes are associated to many phenotypes such as micro-+macrovascular disorder, hypertension and neuropathy. One gene that is located on human chromosome 1q32.1, is synaptotagmin II (SYT2), an integral membrane protein of synaptic vesicles thought to serve as Ca²⁺ sensors in the process of vesicular trafficking and exocytosis. Based on Haploview 4.2, release 27, 13 SNPs located in SYT2 gene were located in 2 main haploblocks (FIG. 2A). The SNPs (rs946857, rs12404969, rs4537626, rs2153441, rs7550433 and rs7517181) in haploblock 1 are associated to micro-+macrovascular disorder, macrovascular disease, MI+angina, MACE, MACE+albuminuria and hypertension. The SNPs (rs10920451, rs10920452, rs4950866, rs4950867, rs2095981 and rs6698441) in haploblock 2 are associated to macrovascular disease and atrial fibrillation while SNP rs4453099 is associated to hypertension. The second gene is SRY (sex determining region Y)-box5 (SOX5). This gene, located on human chromosome 12p12.1, encodes a transcription factor involved in the regulation of embryonic development (cell proliferation) and in the determination of the cell fat (FIG. 3). The SOX5 gene SNP rs12426427 is associated to MACE+albuminuria and micro_+macrovascular disease, the SNP rs2728841 is associated to micro_+macrovascular disease, MI+angina, MACE, MACE+albuminuria and hypertension, the SNP rs7316665 is associated to hypertension, the SNPs rs7963345, rs16926892, rs16927597 and rs4262802 are all associated to atrial fibrillation. The third gene is dystrophin (DMD); essentially described in muscular dystrophies (FIG. 4), is located on human chromosome Xp21.2. The DMD gene SNP rs2692986 is associated to albuminuria+LowCC, micro_+macrovascular complication, microvascular disease, MACE+albuminuria and hypertension. SNP rs17330097 is associated to microvascular disease, rs6527243 is associated to MACE. The SNPs rs5972470, rs1379106 and rs10521991 are associated to hypertension. The group of SNPs rs5928032, rs5928033, rs808549, rs5928038, rs808521, rs808517, rs7884312 and rs7880606 are all associated to atrial fibrillation. The SNP rs5972687 is associated to lowCC, micro_+macrovascular disease and retinopathy. The SNP rs6527237 is associated to neuropathy.

### CONCLUSION

Our results suggest a pleiotropic role of PDE4D, DMD, SOX5 and SYT2 genes in several complications of T2D in patients of Caucasian origin.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions. All publications and patents cited above are incorporated herein by reference.

We claim:

1. A method for predicting a risk of developing a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject affected with type-2 diabetes (T2D), comprising detecting, in a sample obtained from said subject, at least one genetic feature which is
(a) a single nucleotide polymorphism (SNP) having the RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441;
(b) a SNP in linkage disequilibrium to the SNP of (a); or
(c) a single tandem repeat (STR) which is in linkage disequilibrium to the SNP of (a);
wherein the detection of said genetic feature in said subject correlates with said risk of developing at least one of said complication.

2. The method according to claim 1, wherein said genetic feature is at least one SNP having the RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441.

3. The method according to claim 1, comprising detecting a STR of at least one gene which is Phosphodiesterase 4D, cAMP-specific (PDE4D; NCBI Gene ID: 5144), Dystrophin (DMD; NCBI Gene ID: 1756), sex determining region Y (SRY)-box5 (SOX5; NCBI Gene ID: 6660), Synaptotagmin II (SYT2; or NCBI Gene ID: 127833).

4. The method according to claim 1, wherein detection of said genetic feature correlates with increased or reduced risk of developing said complication.

5. A method for predicting a risk of developing a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject affected by type II diabetes, comprising detecting,
in a sample obtained from said subject, at least one single nucleotide polymorphism (SNP) having the RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441; or
one STR found to be in linkage disequilibrium with a gene which is Phosphodiesterase 4D, cAMP-specific (PDE4D; NCBI Gene ID: 5144), Dystrophin (DMD; NCBI Gene ID: 1756), sex determining region Y (SRY)-box5 (SOX5; NCBI Gene ID: 6660), Synaptotagmin II (SYT2; or NCBI Gene ID: 127833),
wherein the detection of said SNP or STR in said subject correlates with said risk of developing said complication.

6. A method for predicting a risk of developing a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject affected by T2D, comprising detecting, in a sample obtained from said subject, at least one (SNP) from at least on gene which is Phosphodiesterase 4D, cAMP-specific (PDE4D; NCBI Gene ID: 5144), Dystrophin (DMD; NCBI Gene ID: 1756), sex determining region Y (SRY)-box5 (SOX5; NCBI Gene ID: 6660), Synaptotagmin II (SYT2; NCBI Gene ID: 127833) or at least one STR found to be in linkage disequilibrium with at least one (SNP) of said gene, wherein the detection of said SNP or STR in said subject correlates with said risk of developing said complication.

7. A method for predicting according to claim 5, wherein said SNP is selected on the basis of its p value of association with said complication(s), allele frequency and/or odds ratio.

8. The method according to claim 5, comprising detecting at least two SNPs.

9. The method according to claim 5, comprising detecting at least three SNPs.

10. The method according to claim 5, comprising detecting more than three SNPs

11. The method according to claim 5, wherein comprising detecting at least one SNP associated with Phosphodiesterase 4D, cAMP-specific (PDE4D; or NCBI Gene ID: 5144).

12. The method of claim 5, wherein the complication is albuminuria and the SNP is rs10051847 (PDE4D).

13. The method of claim 5, wherein the complication is macrovascular disorder and the SNP is rs10051847, rs11951359, rs12657171, rs1077183, rs10514870, rs10066573, rs10068543, rs6450502, rs6864156, rs33927508, rs16889615, rs10471476 (PDE4D); rs5972687, rs2692986 (DMD); rs12426427, rs2728841 (SOX5); rs946857, rs12404969, rs4537626, rs2153441, rs7550433 or rs7517181 (SYT2).

14. The method of claim 5, wherein the complication is MI angina and the SNP is rs10051847, rs11951359, rs12657171, rs1077183 (PDE4D); rs2728841 (SOX5); rs946857, rs12404969, rs4537626, rs2153441, rs7550433 or rs7517181 (SYT2).

15. The method of claim 5, wherein the complication is MACE and the SNP is rs10051847, rs11951359, rs12657171, rs1077183 (PDE4D); rs6527243 (DMD); rs2728841 (SOX5); rs946857, rs12404969, rs2153441, rs7550433 or rs7517181 (SYT2).

16. The method of claim 5, wherein the complication is MACE+albuminuria and the SNP is rs829258, rs10051847, rs11951359, rs10461656, rs12657171, rs1077183, rs10514870, rs10066573, rs10068543, rs6450502, rs6864156 (PDE4D); rs2692986 (DMD); rs12426427, rs2728841 (SOX5); rs2153441, rs7550433 or rs7517181 (SYT2).

17. The method of claim 5, wherein the complication is hypertension and the SNP is rs829258, rs10051847, rs11951359, rs10461656 (PDE4D); rs1379106, rs5972470, rs2692986, rs10521991 (DMD); rs7316665, rs2728841 (SOX5); rs946857, rs12404969, rs4537626, rs2153441, rs7550433, rs7517181, rs4453099 (SYT2).

18. The method of claim 5, wherein the complication is atrial fibrillation and the SNP is rs16889508, rs17528550, rs16889512, rs17723785, rs17780860 (PDE4D); rs5928032, rs5928033, rs808549, rs5928038, rs808521, rs7884312, rs808517, rs7880606 (DMD); rs16927597, rs16926892, rs7963345, rs4262802 (SOX5) or rs6698441 (SYT2).

19. The method of claim 5, wherein the complication is neuropathy and the SNP is rs697076, rs294497, rs294496, rs10514859, rs294494, rs1035321, rs36081664, rs294492, rs893190 (PDE4D) or rs6527237 (DMD).

20. The method of claim 5, wherein the complication is microvascular disorder and the SNP is rs2692986 or rs17330097 (DMD).

21. The method of claim 5, wherein the complication is low creatine clearance and the SNP is rs5972687, rs2692986 (DMD).

22. The method according to claim 5, wherein the complication is retinopathy and the SNP is rs5972687 (DMD).

23. The method according to claim 1, wherein the complication is albuminuria+low creatinine clearance and the SNP rs2692986 (DMD).

24. A kit for predicting a complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject affected by type-2 diabetes (T2D) comprising in one or more packages
(a) an oligonucleotide that specifically hybridizes to a SNP RefSNPID (rs) rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441; or
(b) an oligonucleotide which is the complement of (a);
and one or more reagents for the detection of said oligonucleotide.

25. The kit according to claim 24, comprising one or more reagents for polymerase chain reaction (PCR).

26. The kit of claim 24, further comprising an RNase.

27. The kit of claim 24, which further comprises one or more reagents for isolation of cells from a sample.

28. The kit of claim 24, wherein the oligonucleotide is
(k) an oligonucleotide comprising at least 80% sequence identity to the oligonucleotide having the RefSNP ID which is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441; or
(l) an oligonucleotide which is complimentary to the oligonucleotide of (a).

29. The kit of claim 28, wherein the oligonucleotide is
(a) an oligonucleotide comprising at least 95% sequence identity to the oligonucleotide having the RefSNP ID which is rs10051847, rs11951359, rs2968005, rs12657171, rs1077183, rs10514870, rs10066573, rs10068543, rs6450502, rs6864156, rs33927508, rs16889615, rs10471476, rs829258, rs10461656, rs16889508, rs17528550, rs16889512, rs17723785, rs17780860, rs697076, rs294497, rs294496, rs10514859, rs294494, rs1035321, rs36081664, rs294492, rs893190, rs17330097, rs5972687, rs2692986, rs6527243, rs1379106, rs5972470, rs10521991, rs5928032, rs5928033, rs808549, rs5928038, rs808521, rs7884312, rs808517, rs7880606, rs6527237, rs12426427, rs2728841, rs7316665, rs16927597, rs16926892, rs7963345, rs4262802, rs946857, rs12404969, rs4537626, rs2153441, rs7550433, rs7517181, rs10920451, rs10920452, rs4950866, rs2095981, rs4950867, rs4453099 or rs6698441; or
(b) an oligonucleotide which is complimentary to the oligonucleotide of (a).

30. A method for preventing, treating or reducing the risk of T2D or a T2D related complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy in a subject in need thereof, comprising administrating to said subject a therapeutic agent which
(a) alters the enzymatic activity or levels of a polypeptide encoded by at least one risk gene listed in Tables 1-4; or
(b) alters the expression of at least one polymorphism disclosed in Tables 1-4; or
(c) alters the specific metabolic or other biologically related pathway implicating the risk gene of (a).

31. The method according to claim 30, wherein the therapeutic agent alters the expression of at least one polymorphism disclosed in Tables 1-4.

32. The method according to claim 30, wherein the therapeutic agent is an antisense oligonucleotide or an siRNA.

33. The method according to claim 30, wherein the therapeutic agent alters the levels or enzymatic activity of a polypeptide encoded by at least one risk gene listed in Tables 1-4.

34. The method according to claim 30, wherein the therapeutic agent is an antibody.

35. A method for identifying a subject for preventive therapeutic action, comprising detecting, in a sample obtained from said subject, at least one genetic feature which is
(a) at least one single nucleotide polymorphism (SNP) listed in Tables 1-4;
(b) at least one SNP which is in linkage disequilibrium with at least one SNP of (a); or
(c) short tandem repeat (STR) that is in linkage disequilibrium with at least one SNP of (a), wherein the detection of said genetic feature in said subject correlates with the eligibility of said subject for said preventive therapeutic action.

36. The method according to claim 35, wherein the preventive therapeutic action is for the treatment of a type 2 diabetes related complication which is macrovascular disorder, micro/macrovascular disorder, myocardial infarction/angina, MACE, albuminuria, hypertension, atrial fibrillation, neuropathy, microvascular disorder, low creatinine clearance, retinopathy, low creatinine clearance or nephropathy.


SEQUENCE LISTING (65 sequences)

SEQ ID NO: 1 - DNA, 33 bp, Homo sapiens
atgcattctctattcaytcctgttccttttttt

SEQ ID NO: 2 - DNA, 33 bp, Homo sapiens
ctgtaaacttctatackactttgtaccacccat

SEQ ID NO: 3 - DNA, 33 bp, Homo sapiens
tggtgaaacctggttgkactgaacctttaggag

SEQ ID NO: 4 - DNA, 33 bp, Homo sapiens
tgctgagtgaacacaarcagtaacctatgttct

SEQ ID NO: 5 - DNA, 33 bp, Homo sapiens
catagatcttataaaastttaactgagcattag

SEQ ID NO: 6 - DNA, 33 bp, Homo sapiens
tctatcttttgtcagcragtttcctcagagttc

SEQ ID NO: 7 - DNA, 33 bp, Homo sapiens
ttaacctcggactgccwactttcagattttaca

SEQ ID NO: 8 - DNA, 33 bp, Homo sapiens
tatatgaacatttgtaycattcaactgtctaac

SEQ ID NO: 9 - DNA, 33 bp, Homo sapiens
gttggatctctattaawcctctctaaaatgtca

SEQ ID NO: 10 - DNA, 33 bp, Homo sapiens
ggaagctctgtttgcayaggtaagatttgtcaa

SEQ ID NO: 11 - DNA, 33 bp, Homo sapiens
atcaacacacatttaargcatatcagttttacc

SEQ ID NO: 12 - DNA, 33 bp, Homo sapiens
aatgaatcttgccttargctacataaattacaa

SEQ ID NO: 13 - DNA, 33 bp, Homo sapiens
ttcagtgtgcccagackagaactcatcttccct

SEQ ID NO: 14 - DNA, 33 bp, Homo sapiens
tttacatatatgtaagsacatttggtttatctt

SEQ ID NO: 15 - DNA, 33 bp, Homo sapiens
tggctttgagtttgtaraggtatgcatggcctc

SEQ ID NO: 16 - DNA, 33 bp, Homo sapiens
ctgctacattacctacragtttatcccaatcca

SEQ ID NO: 17 - DNA, 33 bp, Homo sapiens
gagtgaagatggtaaarccagaaagatgtgtcc

SEQ ID NO: 18 - DNA, 33 bp, Homo sapiens
aagaaatggcaagtaaygttaagagacaaaatc

SEQ ID NO: 19 - DNA, 33 bp, Homo sapiens
actgttgcagagctgastatgaagagatgactc

SEQ ID NO: 20 - DNA, 33 bp, Homo sapiens
aactgaatggtctctgratctggtagctaaaag

SEQ ID NO: 21 - DNA, 33 bp, Homo sapiens
ctagatattcacgtgarataggatcattccaca

SEQ ID NO: 22 - DNA, 33 bp, Homo sapiens
ttatcttttcattagarggcaggccattctgac

SEQ ID NO: 23 - DNA, 33 bp, Homo sapiens
ccataaaggatttagaycaaaacagaataaaac

SEQ ID NO: 24 - DNA, 33 bp, Homo sapiens
gaccgatcagaagttgkcaacaggccagtgcta

SEQ ID NO: 25 - DNA, 33 bp, Homo sapiens
gcctcacagttaaggayattttagctattcaag

SEQ ID NO: 26 - DNA, 33 bp, Homo sapiens
catctggtacctgtcayggaatttgataattct

SEQ ID NO: 27 - DNA, 33 bp, Homo sapiens
gcttatatcattgcccrcaaatggaaatgtagc

SEQ ID NO: 28 - DNA, 33 bp, Homo sapiens
tgagaggaacagcatamaaaatgatccgcaaaa

SEQ ID NO: 29 - DNA, 33 bp, Homo sapiens
atggtgacgaatagcaycaatgatgagtacacc

SEQ ID NO: 30 - DNA, 33 bp, Homo sapiens
catgcataaagtgaagrctcctttctgggctga

SEQ ID NO: 31 - DNA, 33 bp, Homo sapiens
aagggcaccacgactcycttggaaaattctttt

SEQ ID NO: 32 - DNA, 33 bp, Homo sapiens
tcaaatacattttaacracttcatcttgtatat

SEQ ID NO: 33 - DNA, 33 bp, Homo sapiens
tagcatggtggctttamgatactcacacatttt

SEQ ID NO: 34 - DNA, 33 bp, Homo sapiens
ctgttctgctaacataycctcaataaatcgatc

SEQ ID NO: 35 - DNA, 33 bp, Homo sapiens
tagataaaagtcatagyaccttacgttgtaatt

SEQ ID NO: 36 - DNA, 33 bp, Homo sapiens
aggtatctggatttcayctgccggttgtaaaag

SEQ ID NO: 37 - DNA, 33 bp, Homo sapiens
tctaaatcatacattgraggaaaatggataaag

SEQ ID NO: 38 - DNA, 33 bp, Homo sapiens
agcaaatgttaaagaawggatgcaacttattta

SEQ ID NO: 39 - DNA, 33 bp, Homo sapiens
ggtgactatatctcagkctccctctttgtagta

SEQ ID NO: 40 - DNA, 33 bp, Homo sapiens
gtcaaaggcatgatagrcagtatagattgcaca

SEQ ID NO: 41 - DNA, 33 bp, Homo sapiens
catacaactgcaggtakctttttcatgtaacac

SEQ ID NO: 42 - DNA, 33 bp, Homo sapiens
ttgtagcttgactgtcyccagcaagaaaatgtc

SEQ ID NO: 43 - DNA, 33 bp, Homo sapiens
aggactagatttaggawatgaacaaaaagctca

SEQ ID NO: 44 - DNA, 33 bp, Homo sapiens
taaattagggatttccwgataggtgatgaaaaa

SEQ ID NO: 45 - DNA, 33 bp, Homo sapiens
ttagtgggcaaagaagrataaggggtaagccac

SEQ ID NO: 46 - DNA, 33 bp, Homo sapiens
acaatacctagcttaawgcctcaaacataataa

SEQ ID NO: 47 - DNA, 33 bp, Homo sapiens
ttagatatcttggcaarggaaaaatgtatgtaa

SEQ ID NO: 48 - DNA, 33 bp, Homo sapiens
tttcacttgtgttatcyaaatgcttcctacctc

SEQ ID NO: 49 - DNA, 33 bp, Homo sapiens
gtgttttgtgttcatgraagaaaaaaacacaca

SEQ ID NO: 50 - DNA, 33 bp, Homo sapiens
taaaaccttctgcaatraaaattaacttattta

SEQ ID NO: 51 - DNA, 33 bp, Homo sapiens
tcatacatgagagacawataataagacctgttc

SEQ ID NO: 52 - DNA, 33 bp, Homo sapiens
gccggttaatggattaygtgtatgagtgggtgg

SEQ ID NO: 53 - DNA, 33 bp, Homo sapiens
aactgaggtaaagagargtaaaataacttgtcc

SEQ ID NO: 54 - DNA, 33 bp, Homo sapiens
gatatatagattcacaygcttacctagagcacc

SEQ ID NO: 55 - DNA, 33 bp, Homo sapiens
acatatgtggagggccsaacctcgtgccacagc

SEQ ID NO: 56 - DNA, 33 bp, Homo sapiens
tgctgtggagtgacacygggggctctttgctga

SEQ ID NO: 57 - DNA, 33 bp, Homo sapiens
ccactgatcaccatgtsaaaatctcctggagca

SEQ ID NO: 58 - DNA, 33 bp, Homo sapiens
ggtgatcagtggaccayctttatttttttattt

SEQ ID NO: 59 - DNA, 33 bp, Homo sapiens
actgtaaaacgtgccayatgatagtttgaaaac

SEQ ID NO: 60 - DNA, 33 bp, Homo sapiens
cattcagtccatagcaytgcatttgtcaattat

SEQ ID NO: 61 - DNA, 33 bp, Homo sapiens
tcattcaggcttctaayggcaaatgagatcaga

SEQ ID NO: 62 - DNA, 33 bp, Homo sapiens
ggaactgtgagttgatkaaatctctcttcttca

SEQ ID NO: 63 - DNA, 33 bp, Homo sapiens
atatacctgtgctctcycattgttcagaaattc

SEQ ID NO: 64 - DNA, 33 bp, Homo sapiens
gtgttcggtctgtctgyaatacattactttgat

SEQ ID NO: 65 - DNA, 33 bp, Homo sapiens
ccaagcatggcttctarcactatagaaaaggtt